(19) United States
(12) Patent Application Publication
(10) Pub. No.: US 2002/0042377 A1
(43) Pub. Date: Apr. 11, 2002

(54) Rotamase enzyme activity inhibitors

(76) Inventors: Joseph P. Steiner, Mount Airy, MD (US); Gregory S. Hamilton, Catonsville, MD (US)

Correspondence Address: FINNEGAN, HENDERSON, FARABOW, GARRETT & DUNNER, L.L.P., 1300 I Street, N.W., Washington, DC, 20005-3315, US

(21) Appl. No.: 09/873,298

(22) Filed: Jun. 5, 2001

Related U.S. Application Data
(63) Continuation-in-part of application No. 08/551,026, filed on Oct. 31, 1995.
(63) Continuation-in-part of application No. 09/359,351, filed on Jul. 21, 1999.
Application No. 09/359,351 is a continuation of application No. 08/693,003, filed on Aug. 6, 1996, now abandoned.
Application No. 08/693,003 is a continuation of application No. 08/479,436, filed on Jun. 7, 1995, now Pat. No. 5,614,547.

(51) Int. Cl. A61K 38/05; A61K 31/221; A61K 31/16

(52) U.S. Cl. 514/19; 514/626; 514/534

(57) ABSTRACT

This invention relates to methods of using neurotrophic compounds having an affinity for FKBP-type immunophilins to stimulate or promote neuronal growth or regeneration and to prevent neuronal degeneration.

This application is a continuation-in-part of U.S. application Ser. No. 08/551,026, filed Oct. 31, 1995, and of U.S. application Ser. No. 09/359,351, filed Jul. 21, 1999, which is a continuation of U.S. application Ser. No. 08/693,003, filed Aug. 6, 1996, which is a continuation of U.S. application Ser. No. 08/479,436, filed Jun. 7, 1995, now U.S. Pat. No. 5,614,547, which are hereby incorporated by reference in their entirety.

This invention relates to neurotrophic compounds having an affinity for FKBP-type immunophilins, their preparation and use as inhibitors of the enzyme activity associated with immunophilin proteins, and particularly inhibitors of peptidyi-prolyl isomerase or rotamase enzyme activity, and their use as small molecule neurotrophic drugs.

The term immunophilin refers to a number of proteins that serve as receptors for the principal immunosuppressant drugs, cyclosporin A (CsA), FK506, and rapamycin. Known classes of immunophilins are cyclophilins and FK506 binding proteins, such as FKBP. Cyclosporin A binds to cyclophilin, while FK506 and rapamycin bind to FKBP. These immunophilin-drug complexes interface with a variety of intracellular signal transduction systems, especially in the immune system and the nervous system.

Immunophilins are known to have peptidyl-prolyl isomerase (PPlase) or rotamase enzyme activity. It has been determined that rotamase activity has a role in the catalyzation of the interconversion of the cis and trans isomer of the substrate proteins of the immunophilin.

Immunophilins were originally discovered and studied in immune tissue. It was initially postulated by those skilled in the art that inhibition of the immunophilin's rotamase activity leads to the inhibition of T-cell proliferation, thereby causing the immunosuppressive action exhibited by immunosuppressive drugs such as cyclosporin A, FK506, and rapamycin. Further study has shown that the inhibition of rotamase activity, in and of itself, is not sufficient for immunosuppressant activity. Instead immunosuppression appears to stem from the formation of a complex of immunosuppressant drugs and immunophilins. It has been shown that the immunophilin-drug complexes interact with ternary protein targets as their mode of action. In the case of FKBP-FK506 and cyclophilin-CsA, the drug-immunophilin complexes bind to the enzyme calcineurin, inhibiting T-cell receptor signaling leading to T-cell proliferation. Similarly, the complex of rapamycin and FKBP interacts with the RAFT11FRAP protein and inhibits signaling from the IL-2 receptor.

Immunophilins have been found to be present at high concentrations in the central nervous system. Immunophilins are enriched 10-50 times more in the central nervous system than in the immune system. Within neural tissues, immunophilins appear to influence nitric oxide synthesis, neurotransmitter release, and neuronal process extension.

FK506 also augments the phosphorylation of growth-associated protein-43 (GAP43). GAP43 is involved in neuronal process extension and its phosphorylation appears to augment this activity. Accordingly, the effects of FK506, rapamycin, and cyclosporin A in neuronal process extension have been examined using PC12 cells. PC12 cells are a continuous line of neuronal-like cells which extend neurites when stimulated by nerve growth factor (NGF).

Surprisingly, it has been found that picomolar concentrations of an immunosuppressant such as FK506 or rapamycin stimulate neurite outgrowth in PC12 cells and sensory neurons, namely dorsal root ganglion cells (DRGs). In whole animal experiments, FK506 has been shown to stimulate nerve regeneration following facial nerve injury and results in functional recovery in animals with sciatic nerve lesions.

More particularly, it has been found that drugs with a high affinity for FKBP are potent rotamase inhibitors and exhibit excellent neurotrophic effects. Snyder et al., “Immunophilins and the Nervous System”, Nature Medicine, Volume 1, No. 1, January 1995, 32-37. These findings suggest the use of immunosuppressants in treating various peripheral neuropathies and in enhancing neuronal regrowth in the central nervous system (CNS). Studies have demonstrated that neurodegenerative disorders such as senile dementia of the Alzheimer's type (Alzheimer's disease, SDAT), Parkinson's disease, and amyotrophic lateral sclerosis (ALS) may occur due to the loss, or decreased availability, of a neurotrophic substance specific for a particular population of neurons affected in the disorder.

Several neurotrophic factors effecting specific neuronal populations in the central nervous system have been identified. For example, it has been hypothesized that Alzheimer's disease results from a decrease or loss of nerve growth factor (NGF). It has thus been proposed to treat SDAT patients with exogenous NGF or other neurotrophic proteins such as brain derived growth factor (BDNF), glial derived growth factor, ciliary neurotrophic factor (CNTF), and neurotropin-3 (NT-3) to increase the survival of degenerating neuronal populations.

Clinical application of these proteins in various neurological disease states is hampered by difficulties in the delivery and bioavailability of large proteins to nervous system targets. By contrast, immunosuppressant drugs with neurotrophic activity are relatively small and display specificity. However, when administered chronically, immunosuppressants exhibit a number of potentially serious side effects including nephrotoxicity, such as impairment of glomerular filtration and irreversible interstitial fibrosis (Kopp et al., 1991, J. Am. Soc. Nephrol. 1:162); neurological deficits, such as involuntary tremors, or non-specific cerebral angina such as non-localized headaches (De Groen et al., 1987, N. Engl. J. Med. 317:861); and vascular hypertension with complications resulting therefrom (Kahan et al., 1989 N. Engi. J. Med. 321: 1725).

The present invention provides non-immunosuppressive neurotrophic compounds having an affinity for FKBP-type immunophilins that are extremely potent in augmenting neurite outgrowth, for promoting neuronal growth, and for facilitating regeneration in various neuropathological situations where neuronal repair can be facilitated. Such neuropathological situations include peripheral nerve damage by physical injury or disease state such as diabetes, physical damage to the central nervous system (spinal cord and brain), brain damage associated with stroke, and neurological disorders relating to neurodegeneration, including Parkinson's disease, Alzheimer's disease, and amyotrophic lateral sclerosis.

### SUMMARY OF THE INVENTION

This invention relates to neurotrophic compounds having an affinity for FKBP-type immunophilins and to methods of using neurotrophic compounds having an affinity for FKBP-type immunophilins.

One embodiment of this invention is neurotrophic compounds of the formula I, detailed below.

Another embodiment of this invention is neurotrophic compounds of the formula II, detailed below.

Another embodiment of this invention is a method of treating a neurological activity in an animal, comprising: administering to an animal an effective amount of a neurotrophic compound having an affinity for FKBP-type immunophilins to stimulate growth of damaged peripheral nerves or to promote neuronal regeneration, wherein the FKBP-type immunophilin exhibits rotamase activity.

Another embodiment of this invention is a method of treating a neurological disorder in an animal, comprising: administering to an animal an effective amount of a neurotrophic compound having an affinity for FKBP-type immunophilins in combination with an effective amount of a neurotrophic factor selected from the group consisting of nerve growth factor, brain derived growth factor, glial derived growth factor, ciliary neurotrophic factor, and neurotrophin-3, to stimulate growth of damaged peripheral nerves or to promote neuronal regeneration, wherein the FKBP-type immunophilin exhibits rotamase activity.

Another embodiment of this invention is a method of stimulating growth of damaged peripheral nerves, comprising: administering to damaged peripheral nerves an effective amount of a neurotrophic compound having an affinity for FKBP-type immunophilins to stimulate or promote growth of the damaged peripheral nerves, wherein the FKBP-type immunophilins exhibit rotamase activity.

Another embodiment of this invention is a method for promoting neuronal regeneration and growth in animals, comprising: administering to an animal an effective amount of a neurotrophic compound having an affinity for FKBP-type immunophilins to promote neuronal regeneration, wherein the FKBP-type immunophilins exhibit rotamase activity.

Yet another embodiment of this invention is a method for preventing neurodegeneration in an animal, comprising: administering to an animal an effective amount of a neurotrophic compound having an affinity for FKBP-type immunophilins to prevent neurodegeneration, wherein the FKBP-type immunophilins exhibit rotamase activity.

### DETAILED DESCRIPTION OF THE INVENTION

The novel neurotrophic compounds of this invention are relatively small molecules in relation to other known compounds, such as rapamycin, FK506, and cyclosporin A.

The neurotrophic compounds of this invention have an affinity for the FK506 binding proteins such as FKBP-12. When the neurotrophic compounds of the invention are bound to FKBP, they have been found to inhibit the prolyl-peptidyl cis-trans isomerase activity, or rotamase activity of the binding protein. The compounds of the invention also have been found to stimulate neurite growth, while not exhibiting an immunosuppressive effect. That is, the compounds of the invention are non-immunosuppresive.

The term “non-immunosuppressive” refers to the inability of the compounds of the present invention to suppress the immune system when compared to a control such as FK506 or cyclosporin A. Assays for determining whether a compound is immunosuppressive are well known to those of ordinary skill in the art. Specific non-limiting examples of well known assays include PMA and OKT3 assays wherein mitogens are used to stimulate proliferation of human peripheral blood lymphocytes (PBC). Compounds added to such assay systems are evaluated for their ability to inhibit such proliferation.

In one embodiment, this invention relates to a novel class of neurotrophic compounds represented by the formula I:

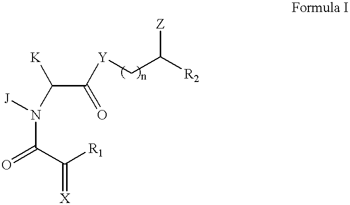

and pharmaceutically acceptable salts thereof,

wherein Y is CH₂, O, NH, or N-(C1-C4 alkyl);

wherein Z and R₂ are independently Ar, (C5-C7)-cycloalkyl substituted (C1-C6)-straight or branched alkyl or (C2-C6)-straight or branched alkenyl, (C5-C7)-cycloalkenyl substituted (C1-C6)-straight or branched alkyl or (C2-C6)-straight or branched alkenyl, or Ar substituted (C1-C6)-straight or branched alkyl or (C2-C6)-straight or branched alkenyl, wherein in each case, one or two carbon atoms of the straight or branched alkyl or alkenyl groups may be substituted with 1-2 heteroatoms selected from the group consisting of oxygen, sulfur, SO and SO₂ in chemically reasonable substitution patterns, or

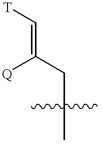

wherein Q is hydrogen, (C1-C6)-straight or branched alkyl or (C2-C6)-straight or branched alkenyl;

wherein T is Ar or substituted 5-7 membered cycloalkyl with substituents at positions 3 and 4 which are independently selected from the group consisting of hydrogen, hydroxyl, O-(C1-C4)-alkyl or O-(C2-C4)-alkenyl and carbonyl;

wherein Ar is selected from the group consisting of monocyclic and bicyclic heterocyclic aromatic ring systems with individual ring sizes being 5 or 6 which may contain in either or both rings a total of 1-4 heteroatoms independently selected from oxygen, nitrogen and sulfur; wherein 1-naphthyl, 2-naphthyl, 2-furyl, 3-furyl, 2-thienyl, 3-thienyl, 2-pyridyl, 3-pyridyl, 4-pyridyl and phenyl are preferred, and wherein Ar may contain one to three substituents which are independently selected from the group consisting of hydrogen, halo, hydroxyl, hydroxymethyl, nitro, CF₃, trifluoromethoxy, (C1-C6)-straight or branched alkyl or (C2-C6)-straight or branched alkenyl, O-(C1-C4)-straight or branched alkyl or O-(C2-C4)-straight or branched alkenyl, O-benzyl, O-phenyl, amino, 1,2-methylenedioxy, carbonyl and phenyl;

wherein R₁ is either hydrogen or U; X is either oxygen or CH-U, provided that if R₁ is hydrogen, then X is CH-U, or if X is oxygen then R₁ is U;

wherein U is hydrogen, O-(C1-C4)-straight or branched alkyl or O-(C2-C4)-straight or branched alkenyl, (C1-C6)-straight or branched alkyl or (C2-C6)-straight or branched alkenyl, (C5-C7)-cycloalkyl, (C5-C7)-cycloalkenyl substituted with (C1-C4)-straight or branched alkyl or (C2-C4)-straight or branched alkenyl, [(C1-C4)-alkyl or (C2-C4)-alkenyl]-Ar or Ar;

wherein J is hydrogen or Cl or C2 alkyl or benzyl; K is (C1-C4)-straight or branched alkyl, benzyl or cyclohexylethyl; or wherein J and K may be taken together to form a 5-7 membered heterocyclic ring which may contain an oxygen (O), sulfur (S), SO or SO₂ substituted therein; and

wherein n is 0-3.

The stereochemistry at position 1 (Formula I) is (R) or (S), with (S) preferred. The stereochemistry at position 2 is (R) or (S).

In a second embodiment, a novel class of neurotrophic compounds of this invention are represented by the formula II:

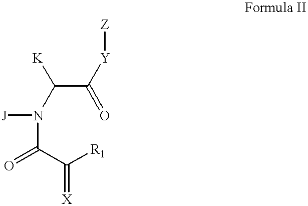

and pharmaceutically acceptable salts thereof,

wherein Y is O, NH, or N-(C1-C4 alkyl);

wherein Z is hydrogen, CHL-Ar, (C1-C6)-straight or branched alkyl, (C2-C6)-straight or branched alkenyl, (C5-C7)-cycloalkyl, (C5-C7)-cycloalkenyl or Ar substituted (C1-C6)-alkyl or (C2-C6)-alkenyl, or

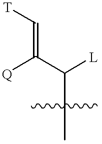

wherein L and Q are independently hydrogen, (C1-C6)-straight or branched alkyl or (C2-C6)-straight or branched alkenyl;

wherein T is Ar or substituted cyclohexyl with substituents at positions 3 and 4 which are independently selected from the group consisting of hydrogen, hydroxyl, O-(C1-C4)-alkyl or O-(C2-C4)-alkenyl and carbonyl;

wherein Ar is selected from the group consisting of 1-naphthyl, 2-naphthyl, 2-furyl, 3-furyl, 2-thienyl, 2-pyridyl, 3-pyridyl, 4-pyridyl and phenyl having one to three substituents which are independently selected from the group consisting of hydrogen, halo, hydroxyl, nitro, CF₃, (C1-C6)-straight or branched alkyl or (C2-C6)-straight or branched alkenyl, O-(C1-C4)-straight or branched alkyl or O-(C2-C4)-straight or branched alkenyl, O-benzyl, O-phenyl, amino and phenyl;

wherein R, is U; X is either oxygen or CH-U, provided that if R, is hydrogen, then X is CH-U, or if X is oxygen then R, is U;

wherein U is hydrogen, O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar;

wherein J is hydrogen or C1 or C2 alkyl or benzyl; K is (C1-C4)-straight or branched alkyl, benzyl or cyclohexylethyl; or wherein J and K may be taken together to form a 5-7 membered heterocyclic ring which may contain an oxygen (O), sulfur (S), SO or SO₂ substituted therein.

The stereochemistry at position 1 (Formula II) is (R) or (S), with (S) preferred.

The compounds of this invention exist as stereoisomeric forms, either as enantiomers or diastereoisomers. The stereochemistry at position 1 of Formula I or II is R or S, with S preferred. Included within the scope of the invention are the enantiomers, the racemic form, and the diastereoisomeric mixtures. Enantiomers as well as diastereoisomers can be separated by methods known to those skilled in the art.

It is known that immunophilins such as FKBP preferentially recognize peptide substrates containing Xaa-Pro-Yaa motifs, where Xaa and Yaa are lipophilic amino acid residues. Schreiber et al. 1990 J. Org. Chem. 55, 4984-4986; Harrison and Stein, 1990 Biochemistry, 29, 3813-3816. Thus, modified prolyl peptidomimetic compounds bearing lipophilic substituents should bind with high affinity to the hydrophobic core of the FKBP active site and inhibit its rotamase activity.

The compounds of the present invention can be used in the form of salts derived from inorganic or organic acids and bases. Included among such acid salts are the following: acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemissulfate heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, propionate, succinate, tartrate, thiocyanate, tosylate and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups can be quarternized with such agents as lower alkyl halides, such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; aralkyl halides like benzyl and phenethyl bromides; and others. Water or oil-soluble or dispersible products are thereby obtained.

The neurotrophic compounds of this invention can be periodically administered to a patient undergoing treatment for neurological disorders or for other reasons in which it is desirable to stimulate neuronal regeneration and growth, such as in various peripheral neuropathic and neurological disorders relating to neurodegeneration. The compounds of this invention can also be administered to animals, including mammals other than humans, for treatment of various neurological disorders.

The novel compounds of the present invention are potent inhibitors of rotamase activity and possess an excellent degree of neurotrophic activity. The neurotrophic activity is useful in the stimulation of growth of damaged neurons, the promotion of neuronal regeneration, the prevention of neurodegeneration, and in the treatment of several neurological disorders known to be associated with neuronal degeneration and peripheral neuropathies. The neurological disorders that may be treated include, but are not limited to: trigeminal neuralgia, glossopharyngeal neuralgia, Bell's Palsy, myasthenia gravis, muscular dystrophy, amyotrophic lateral sclerosis, progressive muscular atrophy, progressive bulbar inherited muscular atrophy, herniated, ruptured or prolapsed invertabrae disk syndromes, cervical spondylosis, plexus disorders, thoracic outlet destruction syndromes, peripheral neuropathies such as those caused by lead, dapsone, ticks, porphyria, or Guillain-Barre syndrome, Alzheimer's disease, and Parkinson's disease.

For these purposes the compounds of the present invention may be administered orally, parenterally, by inhalation spray, topically, rectally, nasally, buccally, vaginally or via an implanted reservoir in dosage formulations containing conventional non-toxic pharmaceutically-acceptable carriers, adjuvants and vehicles. The term parenteral as used herein includes subcutaneous, intravenous, intramuscular, intraperitoneal, intrathecal, intraventricular, intrasternal and intracranial injection or infusion techniques.

To be effective therapeutically for the treatment of CNS disorders, the compounds of the invention should be able to readily penetrate the blood-brain barrier. Compounds of this invention which cannot penetrate the blood-brain barrier can be effectively administered by an intraventricular route.

The pharmaceutical compositions may be in the form of a sterile injectable preparation, for example as a sterile injectable aqueous or oleaginous suspension. This suspension may be formulated according to techniques know in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. Fatty acids such as oleic acid and its glyceride derivatives find use in the preparation of injectables, as do olive oil or castor oil, especially in their polyoxyethylated versions. These oil solutions or suspensions may also contain a long-chain alcohol diluent or dispersant.

The compounds may be administered orally in the form of capsules or tablets, for example, or as an aqueous suspension or solution. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening and/or flavoring and/or coloring agents may be added.

The compounds of this invention may also be administered in the form of suppositories for rectal administration of the drug. These compositions can be prepared by mixing the drug with a suitable non-irritating excipient which is solid at room temperature but liquid at rectal temperature and therefore will melt in the rectum to release the drug. Such materials include cocoa butter, beeswax and polyethylene glycols.

The compounds of this invention may also be administered topically, especially when the conditions addressed for treatment involve areas or organs readily accessible by topical application, including neurological disorders of the eye, the skin, or the lower intestinal tract. Suitable topical formulations are readily prepared for each of these areas.

For ophthalmic use, the compounds can be formulated as micronized suspensions in isotonic, pH adjusted sterile saline, or, preferably, as solutions is isotonic, pH adjusted sterile saline, either with or without a preservative such as benzylalkonium chloride. Alternatively for the ophthalmic uses the compounds may be formulated in an ointment such as petrolatum.

For application topically to the skin, the compounds can be formulated in a suitable ointment containing the compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, the compounds can be formulated in a suitable lotion or cream containing the active compound suspended or dissolved in, for example, a mixture of one or more of the following: mineral oil, sorbitan monostearate, polysorbate 60, cetyl esters wax, cetearyl alcohol, 2-octyldodecanol, benzyl alcohol and water.

Topical application for the lower intestinal tract can be effected in a rectal suppository formulation (see above) or in a suitable enema formulation.

Dosage levels on the order of about 0.1 mg to about 10,000 mg of the active ingredient compound are useful in the treatment of the above conditions, with preferred levels of about 0.1 mg to about 1,000 mg. The amount of active ingredient that may be combined with the carrier materials to produce a single dosage form will vary depending upon the host treated and the particular mode of administration.

It is understood, however, that a specific dose level for any particular patient will depend upon a variety of factors including the activity of the specific compound employed, the age, body weight, general health, sex, diet, time of administration, rate of excretion, drug combination, and the severity of the particular disease being treated and form of administration.

The compounds can be administered with other neurotrophic agents such as nerve growth factor (NGF), glial derived growth factor, brain derived growth factor, ciliary neurotrophic factor, and neurotropin-3. The dosage level of other neurotrophic drugs will depend upon the factors previously stated and the neurotrophic effectiveness of the drug combination.

### Methods and Procedures Ki Test Procedure

Inhibition of the peptidyl-prolyl isomerase (rotamase) activity of the inventive compounds can be evaluated by known methods described in the literature (Harding, M. W. et al. Nature 341: 758-760 (1989); Holt et al. J. Am. Chem. Soc. 115: 9923-9938). These values are obtained as apparent Kᵢ values and are presented in Table I. The cis-trans isomerization of an phenylalanine-proline bond in a model substrate, N-succinyl-Ala-Phe-Pro-Phe-p-nitroanilide, is monitored spectrophotometrically in a chymotrypsin-coupled assay, which releases para-nitroanilide from the trans form of the substrate. The inhibition of this reaction caused by the addition of different concentrations of inhibitor is determined, and the data is analyzed as a change in first-order rate constant as a function of inhibitor concentration to yield the apparent Kᵢ values.

In a plastic cuvette are added 950 μL of ice cold assay buffer (25 mM HEPES, pH 7.8, 100 mM NaCG), 10 μL of FKBP (2.5 μM in 10 mM Tris-Cl pH 7.5, 100 mM NaCl, 1 mM dithiothreitol), 25 μL of chymotrypsin (50 mg/ml in 1 mM HCl) and 10 μL of test compound at various concentrations in dimethyl sulfoxide. The reaction is initiated by the addition of 5 μL of substrate (succinyl-Ala-Phe-Pro-Phe-para-nitroanilide, 5 mg/mL in 2.35 mM LiCl in trifluoroethanol).

The absorbance at 390 nm versus time is monitored for 90 sec using a ctrophotometer and the rate constants are determined from the absorbance versus e data files.

The data for these experiments are presented in Table I and in Table II.

TABLE I

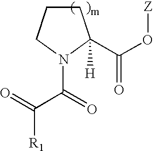

| No. | Z | R₁ | m | Kᵢ μM |
|---|---|---|---|---|
| 1 | Benzyl | Phenyl | 2 | 1.5 |
| 2 | 3-Phenylpropyl | Phenyl | 2 | — |
| 3 | 4-(4-Methoxy- | Phenyl | 2 | — |
| | phenyl(butyl | | | |
| 4 | 4-Phenylbutyl | Phenyl | 2 | 0.35 |
| 5 | Phenethyl | Phenyl | 2 | 1.1 |
| 6 | 4-Cyclohexyl- | Phenyl | 2 | 0.4 |
| | butyl | | | |
| 7 | Benzyl | Methoxy | 2 | 80 |
| 8 | 4-Cyclohexyl- | Methoxy | 2 | 6 |
| | butyl | | | |
| 9 | 3-Cyclohexyl- | Methoxy | 2 | 20 |
| | propyl | | | |
| 10 | 3-Cyclopentyl- | Methoxy | 2 | 35 |
| | propyl | | | |
| 11 | Benzyl | 2-Furyl | 2 | 3 |
| 12 | 4-Cyclohexyl- | 3,4,5-Trimethoxy- | 2 | 0.04 |
| | butyl | phenyl | | |
| 13 | 3-Phenoxy- | 3,4,5-Trimethoxy- | 2 | 0.018 |
| | benzyl | phenyl | | |
| 14 | 4-Phenylbutyl | 3,4,5-Trimethoxy- | 2 | 0.019 |
| | | phenyl | | |
| 15 | 3-(3-indolyl) | 3,4,5-Trimethoxy- | 2 | 0.017 |
| | propyl | phenyl | | |
| 16 | 4-(4-Methoxy- | 3,4,5-Trimethoxy- | 2 | 0.013 |
| | phenyl)butyl | phenyl | | |
| 17 | 3-phenyl-1-propyl | 1,1-dimethylpropyl | 1 | 0.042 |
| 18 | 3-phenyl-1-prop- | 1,1-dimethylpropyl | 1 | 0.125 |
| | 2-(E)-enyl | | | |
| 19 | 3-(3,4,5-trimethoxy- | 1,1-dimethylpropyl | 1 | 0.025 |
| | phenyl)-1-propyl | | | |
| 20 | 3-(3,4,5-trimethoxy- | 1,1-dimethylpropyl | 1 | 0.125 |
| | phenyl)-1-prop-2- | | | |
| | (E)-enyl | | | |
| 21 | 3-(4,5-dichloro- | 1,1-dimethylpropyl | 1 | 2.50 |
| | phenyl)-1-prop-2- | | | |
| | (E)-enyl | | | |
| 22 | 3-(2,5-dimethoxy- | 1,1-dimethylpropyl | 1 | 0.450 |
| | phenyl)-1-prop-2- | | | |
| | (E)-enyl | | | |
| 23 | 3-(3-pyridyl)-1- | 1,1-dimethylpropyl | 1 | 0.0075 |
| | propyl | | | |
| 24 | 3-phenyl-1-propyl | cyclohexyl | 1 | 0.082 |

TABLE II

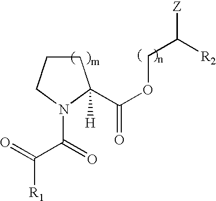

| | | | | | | Kᵢ |
|---|---|---|---|---|---|---|
| No. | m | n | Z | R₂ | R₁ | nM |
| 25 | 2 | 0 | 3-Phenyl- | 3-(3-Pyridyl) | Phenyl | 56 |
| | | | propyl | propyl | | |
| 26 | 2 | 0 | 3-Phenyl- | 3-(2-Pyridyl) | Phenyl | 50 |
| | | | propyl | propyl | | |
| 27 | 2 | 0 | 3-Phenyl- | 2-(4-Methoxy- | Phenyl | 270 |
| | | | propyl | phenyl)ethyl | | |
| 28 | 2 | 0 | 3-Phenyl- | 3-Phenylpropyl | Phenyl | — |
| | | | propyl | | | |
| 29 | 2 | 0 | 3-Phenyl- | 3-Phenylpropyl | 3,4,5-Trimeth- | 1.0 |
| | | | propyl | | oxyphenyl | |
| 30 | 2 | 0 | 3-Phenyl- | 2-(3-Pyridyl) | 3,4,5-Trimeth- | 3.0 |
| | | | propyl | | oxyphenyl | |
| 31 | 2 | 0 | 3-Phenyl- | 3-(2-Pyridyl) | 3,4,5-Trimeth- | 1.0 |
| | | | propyl | | oxyphenyl | |
| 32 | 2 | 0 | 3-Phenyl- | 3-(4-Methoxy- | 3,4,5-Trimeth- | −3.0 |
| | | | propyl | phenyl)propyl | oxyphenyl | |
| 33 | 2 | 0 | 3-Phenyl- | 3-(3-Pyridyl) | 3-Iso-propoxy- | 2.0 |
| | | | propyl | propyl | phenyl | |
| 34 | 1 | 1 | 3-pyridyl | 3-phenyl | 1,1-dimethylpropyl | 0.019 |

### Chick Dorsal Root Ganglion Cultures and Neurite Outgrowth

Dorsal root ganglia were dissected from chick embryos of ten day gestation. Whole ganglion explants were cultured on thin layer Matrigel-coated 12 well plates with Liebovitz L15 plus high glucose media supplemented with 2 mM glutamine and 10% fetal calf serum, and also containing 10 pM cytosine P-D arabinofuranoside (Ara C) at 37° C. in an environment containing 5% CO₂. Twenty-four hours later, the DRGs were treated with various concentrations of nerve growth factor (NGF), immunophilin ligands or combinations of NGF plus drugs. Forty-eight hours after drug treatment, the ganglia were visualized under phase contrast or Hoffman Modulation contrast with a Zeiss Axiovert inverted microscope. Photomicrographs of the explants were made, and neurite outgrowth was quantitated. Neurites longer than the DRG diameter were counted as positive, with total number of neurites quantitated per each experimental condition. Three to four DRGs were cultured per well, and each treatment was performed in duplicate.

The data for the drug alone (e.g., immunophilin ligand) experiments are presented in Table III.

TABLE III
Neurite Outgrowth in Chick DRG

| | ED₅₀, nM |
|---|---|
| Compound | Neurite Outgrowth in DRG Cultures |
| 1 | 25-100 |
| 2 | 10-20 |
| 3 | 0.500 |
| 4 | 25-100 |
| 5 | 25-100 |
| 6 | 10-20 |
| 7 | >10,000 |
| 8 | >10,000 |
| 9 | >10,000 |
| 10 | >10,000 |
| 11 | 1000 |
| 12 | 0.031 |
| 13 | 0.180 |
| 14 | 1-5 |
| 15 | 0.055 |
| 16 | 0.030 |
| 17 | 0.053 |
| 18 | 105 |
| 19 | 80 |
| 20 | 190 |
| 21 | 85 |
| 22 | 0.8 |
| 23 | 0.05 |
| 24 | 0.13 |
| 25 | 1-5 |
| 26 | 0.063 |
| 27 | 10-20 |
| 28 | 0.0044 |
| 29 | 0.61 |
| 30 | 0.95 |
| 31 | 25 |
| 32 | 0.50 |
| 33 | 0.30 |
| 34 | 0.07 |

### EXAMPLES

The inventive compounds may be prepared by a variety of synthetic sequences that utilize established chemical transformations. The general pathway to the present compounds is described in Scheme 1. N-Glyoxylproline derivatives may be prepared by reading L-proline methyl ester with methyl oxalyl chloride as shown in Scheme I. The resulting oxamates may be reacted with a variety of carbon nucleophiles to obtain intermediate compounds. These intermediates are then reacted with a variety of alcohols, amides, or protected amino acid residues to obtain the prolyl esters, ketones, acids, and amides of the invention.

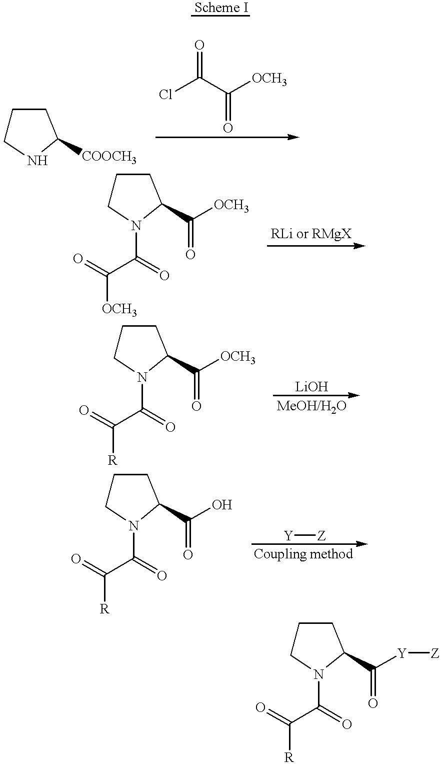

### Example 1

Synthesis of methyl (2S)-1-(1,2-dioxo-2-methoxyethyl)-2-pyrrolidinecarboxylate.

A solution of L-proline methyl ester hydrochloride (3.08 g; 18.60 mmol) in dry methylene chloride was cooled to 0° C. and treated with triethylamine (3.92 g; 38.74 mmol; 2.1 eq). After stirring the formed slurry under a nitrogen atmosphere for 15 min, a solution of methyl oxalyl chloride (3.20 g; 26.12 mmol) in methylene chloride (45 mL) was added dropwise. The resulting mixture was stirred at 0° C. for 1.5 hr. After filtering to remove solids, the organic phase was washed with water, dried over MgSO₄ and concentrated. The crude residue was purified on a silica gel column, eluting with 50% ethyl acetate in hexane, to obtain 3.52 g (88%) of the product as a reddish oil. Mixture of cis-trans amide rotamers; data for trans rotamer given. ¹H NMR (CDCl₃): δ1.93 (dm, 2H); 2.17 (m, 2H); 3.62 (m, 2H); 3.71 (s, 3H); 3.79, 3.84 (s, 3H total); 4.86 (dd, 1H, J=8.4, 3.3).

### Example 2

General procedure for the synthesis of pyrrolidinyl alkyl oxamates. Exemplified for methyl (2S)-1-(1,2-dioxo-3,3-dimethylpentyl)-2-pyrrolidinecarboxylate.

A solution of methyl (2S)-1-(1,2-dioxo-2-methoxyethyl)-2-pyrrolidinecarboxylate (2.35 g; 10.90 mmol) in 30 mL of tetrahydrofuran (THF) was cooled to −78° C. and treated with 14.2 mL of a 1.0 M solution of 1,1-dimethylpropylmagnesium chloride in THF. After stirring the resulting homogenous mixture at −780C for three hours, the mixture was poured into saturated ammonium chloride (100 mL) and extracted into ethyl acetate. The organic phase was washed with water, dried, and concentrated, and the crude material obtained upon removal of the solvent was purified on a silica gel column, eluting with 25% ethyl acetate in hexane, to obtain 2.10 g (75%) of the oxamate as a colorless oil. ¹H NMR (CDCl₃): δ0.88 (t, 3H); 1.22, 1.26 (s, 3H each); 1.75 (dm, 2H); 1.87-2.10 (m, 3H); 2.23 (m, 1H); 3.54 (m, 2H); 3.76 (s, 3H); 4.52 (dm, 1H, J-8.4, 3.4).

### Example 3

General procedure for the preparation of pyrrolidine carboxylic acids. Exemplified for (2S)-1-(1,2-dioxo-3,3-d imethylpentyl )-2-pyrrolid inecarboxylic acid A mixture of methyl (2S)-1-(1,2-dioxo-3,3-dimethylpentyl)-2-pyrrolidinecarboxylate (2.10 g; 8.23 mmol), 1 N LiOH (15 mL), and methanol (50 mL) was stirred at 0° C. for 30 min and at room temperature overnight. The mixture was acidified to pH 1 with 1 N HCl, diluted with water, and extracted into 100 mL of methylene chloride. The organic extract was washed with brine and concentrated to deliver 1.73 g (87%) of snow-white solid which did not require further purification. ¹H NMR (CDCl₃): δ0.87 (t, 3H); 1.22,1.25 (s, 3H each); 1.77 (dm, 2H); 2.02 (m, 2H); 2.17 (m, 1H); 2.25 (m, 1H); 3.53 (dd, 2H, J=10.4, 7.3); 4.55 (dd, 1H, J=8.6, 4.1).

### EXAMPLE4

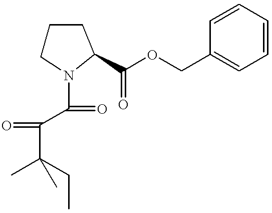

Benzyl (2S)-1-(3,3-dimethyl-1 ,2-dioxopentyl )-2-pyrrolidinecarboxylate.

A mixture of (2S)-1-(1,2-dioxo-3,3-dimethylpentyl)-2-pyrrolidine-carboxylic acid (500 mg; 2.07 mmol), benzyl alcohol (335 mg; 3.10 mmol), dicyclohexylcarbodiimide (683 mg; 3.31 mmol), 4-dimethylaminopyridine (84 mg; 0.69 mmol) and camphorsulphonic acid (160 mg; 0.69 mmol) in methylene chloride (30 mL) was stirred overnight under a nitrogen atmosphere. The reaction mixture was filtered through Celite to remove solids and concentrated in vacuo, and the crude material was purified on a flash column (25% ethyl acetate in hexane) to obtain 680 mg of the product as a colorless oil. ¹H NMR (CDCl₃; 300 MHz): δ0.85 (t, 3H); 1.19 (s, 3H); 1.22 (s, 31); 1.61-2.25 (m, 6H); 3.46-3.56 (m, 2H); 4.58 (dm, 1H); 5.18 (d, 2H, 7.35 (br, 5H).

### Example 5

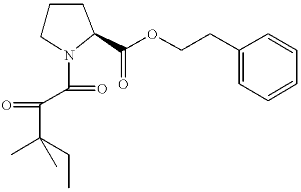

2-Phenyl-1-ethyl (2S)-1-(3,3-dimethyl-1,2-dioxopentyl)-2-pyrrolidinecarboxylate.

A mixture of (2S)-1-(1,2-dioxo-3,3-dimethylpentyl)-2-pyrrolidine-carboxylic acid (570 mg; 2.36 mmol), phenethyl alcohol (432 mg; 3.54 mmol), dicyclohexylcarbodiimide (780 mg; 3.78 mmol), 4-dimethylaminopyridine (98 mg; 0.79 mmol) and amphorsulphonic acid (183 mg; 0.79 mmol) in methylene chloride (30 mL) was stirred overnight under a nitrogen atmosphere. The reaction mixture was filtered through Celite to remove solids and concentrated in vacuo, and the crude material was purified on a flash column (25% ethyl acetate in hexane) to obtain 600 mg of the product as a colorless oil. ¹H NMR (CDCl₃; 300 MHz): δ0.87 (t, 3H); 1.21 (s, 3H); 1.25 (s, 3H); 1.64-1.94 (m, 6H); 2.17 (m, 1H); 2.97 (m, 2H); 3.49 (m, 2H); 4.36 (m, 2H); 4.51 (m, 1H);

7.22-7.33 (m, 5H).

### Example 6

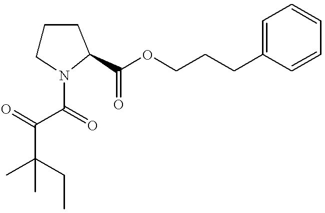

General procedure for the synthesis of prolyl esters. Exemplified for 3-phenyl-1-propyl (2S)-1l-(3 ,3-dimethyl-1,2-dioxopentyl)-2-pyrrolidinecarboxylate. A mixture of (2S)-1-(1 ,2-dioxo-3,3-dimethylpentyl)-2-pyrrolidine-carboxylic acid (600 mg; 2.49 mmol), 3-phenyl-1-propanol (508 mg; 3.73 mmol), dicyclohexylcarbodiimide (822 mg; 3.98 mmol), camphorsulphonic acid (190 mg; 0.8 mmol) and 4-dimethylaminopyridine (I100 mg; 0.8 mmol) in methylene chloride (20 mL) was stirred overnight under a nitrogen atmosphere. The reaction mixture was filtered through Celite to remove solids and concentrated in vacuo, and the crude material was purified on a flash column (25% ethyl acetate in hexane) to obtain 720 mg (80%) of Example 6 as a colorless oil. ¹H NMR (CDCl₃; 300 MHz): δ0.84 (t, 3H); 1.19 (s, 3H); 1.23 (s, 3H); 1.70 (dm, 2H); 1.98 (m, 5H); 2.22 (m, 1H); 2.64 (m, 2H); 3.47 (m, 2H); 4.14 (m, 2H); 4.51 (d, 1H); 7.16 (m, 3H); 7.26 (m, 2H). Example 6 is compound 17 in Tables I and III.

### Example 7

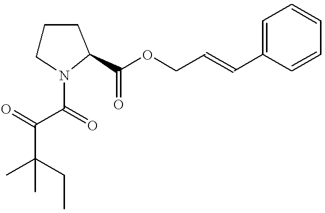

3-Phenyl-l1-prop-2-(E)-enyl (2S)-1l-(3 ,3-dimethyl-1 ,2-dioxopentyl)-2-pyrrolidinecarboxylate, 80%, ¹H NMR (ODC1₃; 360 MHz): δ0.86 (t, 3H); 1.21 (s, 3H); 1.25 (s, 3H); 1.54-2. 10 (m, 5H); 2.10-2.37 (m, 1H); 3.52-3.55 (m, 2H); 4.56 (dd, 1H, J=3.8, 8.9); 4.78-4.83 (m, 2H); 6.27 (m, 1H); 6.67 (dd, 1H, J=15.9); 7.13-7.50 (m, 5H). This compound was prepared by the method of Example 6 from (2S)-l1-(1 ₇2-dioxo-3,3-dimethylpentyl)-2-pyrrolidine-carboxylic acid. Example 7 is compound 18 in Tables I and III.

### Example 8

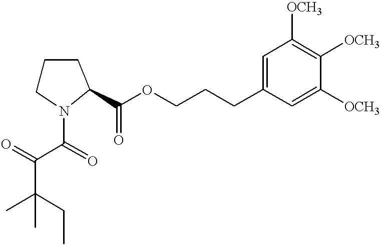

3-(3,4,5-Trimethoxyphenyl)-1-propyl (2S)-1-(3,3-dimethyl-1,2-dioxopentyl)-2-pyrrolidine- carboxylate, 61%, ¹H NMR (CDCl₃; 300 MHz): δ0.84 (t, 3H); 1.15 (s, 3H); 1.24 (s, 3H); 1.71 (dm, 2H); 1.98 (m, 5H); 2.24 (m, 1H); 2.63 (m, 2H); 3.51 (t, 2H); 3.79 (s, 3H); 3.83 (s, 3H); 4.14 (m, 2H); 4.52 (m, 1H); 6.36 (s, 2H). This compound was prepared by the method of Example 8 from (2S)-1-(1,2-dioxo-3,3-dimethylpentyl)-2-pyrrolidine-carboxylic acid. Example 8 is compound 19 in Tables I and III.

### Example 9

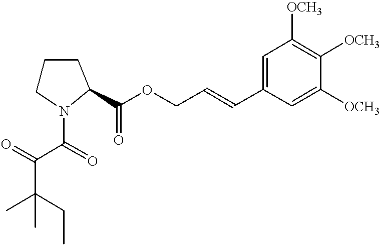

3-(3,4,5-Trimethoxyphenyl)-1-prop-2-(E)-enyl (2S)-1-(3,3-dimethyl-1,2-dioxopentyl)-2-pyrrolidine carboxylate, 66%, ¹H NMR (CDCl₃; 360 MHz): δ0.85 (t, 3H); 1.22 (s, 3H); 1.25 (s, 3H); 1.50-2.11 (m, 5H); 2.11-2.40 (m, 1H); 3.55 (m, 2H); 3.85 (s, 3H); 3.88 (s, 6H); 4.56 (dd, 1H); 4.81 (m, 2H); 6.22 (m, 1H); 6.58 (d, 1H, J=16); 6.63 (s, 2H). This compound was prepared by the method of Example 8 from (2S)-1-(1,2-dioxo-3,3-dimethylpentyl)-2-pyrrolidine-carboxylic acid. Example 9 is compound 20 in Tables I and III.

### Example 10

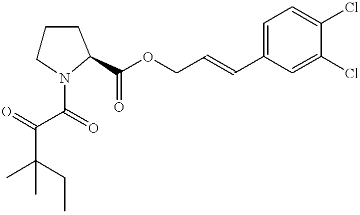

3-(4,5-Dichlorophenyl)-1-prop-2-(E)-enyl (2S)-1-(3,3-d imethyl-1,2-d ioxopentyl )-2-pyrrolidine carboxylate, 70%, ¹H NMR (CDCl₃; 360 MHz): δ0.85 (t, 3H); 1.21 (s, 3H); 1.25 (s, 3H); 1.51-1.87 (m, 2H); 1.87-2.39 (m, 4H); 3.51-3.57 (m, 2H); 4.50-4.61 (dd, 1H, J-3.4, 8.6); 4.80 (d, 2H, J=6.0); 6.20-6.34 (m, 1H); 6.50-6.66 (d, 1H, J=16); 7.13-7.24 (dd, 1H, J=1.8, 8.3); 7.39 (d,1H, J=8.3); 7.47 (s, 1H). This compound was prepared by the method of Example 8 from (2S)-l-(1,2-dioxo-3,3-dimethylpentyl)-2-pyrrolidine-carboxylic acid. Example 10 is compound 21 in Tables I and III.

### Example 11

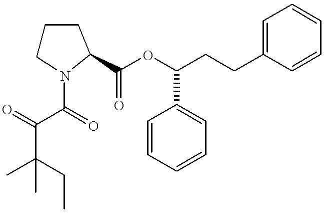

(1R)-1,3-Diphenyl-1-propyl (2S)-1-(3,3-dimethyl-1,2-dioxopentyl)-2-pyrrolidinecarboxylate, 90%, ¹H NMR (CDCl₃; 360 MHz): δ0.85 (t, 3H); 1.20 (s, 3H); 1.23 (s, 3H); 1.49-2.39 (m, 7H); 2.46-2.86 (m, 2H); 3.25-3.80 (m, 2H); 4.42-4.82 (m, 1H); 5.82 (td,1H, J=1.8,6.7); 7.05-7.21 (m, 3H); 7.21-7.46 (m, 7H). This compound was prepared by the method of Example 8 from (2S)-1-(1,2-dioxo-3,3-dimethylpentyl)-2-pyrrolidine-carboxylic acid.

### Example 12

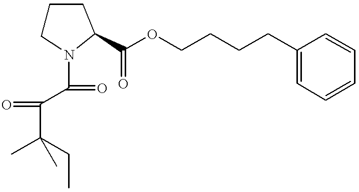

4-Ph enyl-1-butyl (2S)-1l-(3 ,3-dimethyl-1I,2-d ioxopentyl)-2-pyrrolidinecarboxylate.

¹H NMR (CDCl₃; 300 MHz): δ0.84 (t, 3H); 1.22 (s, 3H); 1.25 (s, 3H); 1.64-2.01 (m, 9H); 2.23 (m, 1H); 2.64 (m, 2H); 3.48-3.53 (m, 2H); 4.17 (m, 2H); 4.52 (m, 1H); 7.18 (m, 3H); 7.27 (m, 2H).

### Example 13

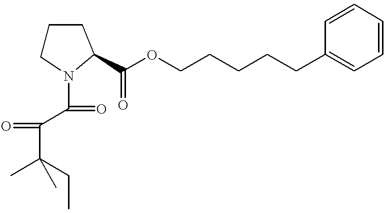

5-Phenyl-1-pentyl (2S)-1-(3,3-dimethyl-1,2-dioxopentyl)-2-pyrrolidinecarboxylate. ¹H NMR (CDCl₃; 300 MHz): δ0.87 (t, 3H); 1.22 (s, 3H); 1.25 (s, 3H); 1.39 (m, 2H); 1.63-1.99 (m, 9H); 2.22 (m, 1H); 2.64 (m, 2H); 3.46-3.54 (m, 2H); 4.14 (m, 2H); 4.50 (m, 1H); 7.16 (m, 3H); 7.26 (m, 2H).

Examples 12 and 13 were prepared according to the synthetic procedure outlined for Examples 4-6, except that the requisite phenyl alcohols in the reaction mixture were 4-phenylbutan-1-ol and 5-phenylpentan-1-ol, respectively.

### Example 14

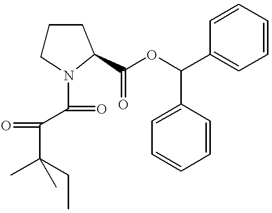

1,1-Diphenylmethyl (2S)-1-(3,3-dimethyl-1 ,2-dioxopentyl)-2-pyrrolidinecarboxylate.

¹H NMR (CDCl₃; 300 MHz): δ0.84 (t, 3H); 1. 17 (s, 3H); 1. 19 (s, 3H); 1.54-2.25 (m, 6H); 3.50 (m, 2H); 4.67 (m, 1H); 5.86 (s, 1H); 7.28-7.39 (m, 10OH).

### Example 15

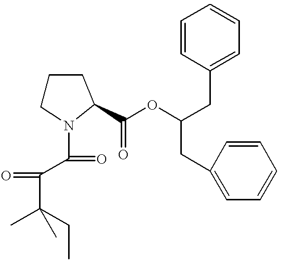

1,3-Diphenyl-2-propyl (2S)-1-(3,3-d imethyl-1,2-d ioxopentyl )-2-pyrrolidinecarboxylate.

¹H NMR (CDCl₃; 300 MHz): δ0.87 (t, 3H); 1.20 (s, 3H); 1.24 (s, 3H); 1.25-2.02 (m, 6H); 2.74, 2.84 (m, 4H total); 3.53 (m, 2H); 4.04 (m, 1H); 4.42 (m,1H); 7.22 (m, 6H); 7.30 (m, 4H).

### Example 16

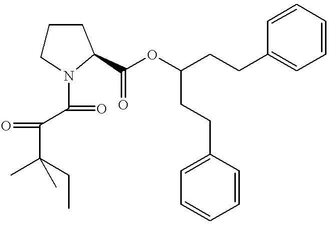

1,5-Diphenyl-3-pentyl (2S)-1-(3,3-dimethyl-1,2-dioxopentyl)-2-pyrrolidinecarboxylate.

¹H NMR (CDCl₃; 300 MHz): δ0.87 (t, 3H); 1.23 (s, 3H); 1.27 (s, 3H); 1.61-2.06 (m, 9H); (m, 2H); 2.28 (m, 1H); 2.57-2.74 (m, 4H); 3.52-3.56 (m, 2H); 4.49-4.59 (m, 1H); 5.02 (m, 1H); 7.14-7.30 (m, 10H).

### Example 17

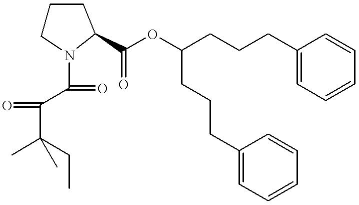

1,7-Diphenyl-4-heptyl (2S)-1l-(3,3-dimethyl-1 ,2-dioxopentyl)-2-pyrrolid inecarboxylate.

¹H NMR (CDCl₃; 300 mHZ): δ0.86 (t, 3H); 1.23 (s, 3H); 1.25 (s, 3H); 1.44-1.98 (m, 13H); 2.21 (m, 1H); 2.59 (m, 4H); 3.45-3.63 (m, 2H); 4.484.52 (dd, 1H, J=2.7, 6.5); 4.99 (m, 1H); 7.08-7.18 (m, 6H); 7.21-7.29 (m, 4H).

Examples 14-17 were prepared according to the synthetic procedure outlined for

Examples 4-6, except that the requisite diphenyl alcohols in the reaction mixture were 1,1-diphenylmethanol, 1,3-diphenylpropan-2-ol, 1,5-diphenylpentan-3-ol, and 1,7-diphenylheptan-4-ol, respectively.

### Example 18

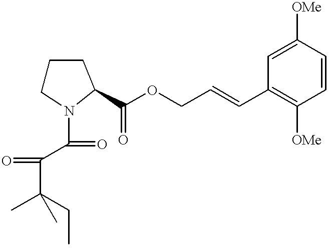

3-(2,5-Dimethoxyphenyl)-1l-prop-2(E)-enyl (2S)-1l-(3,3-dimethyl-1,2-dioxopentyl)-2-pyrrolidinecarboxylate.

¹H NMR (CDCl₃; 300 MHz): δ0.87 (t, 3H); 1.22 (s, 3H); 1.26 (s, 3H); 1.67 (m, 2H); 1.78 (m, 1H); 2.07 (m, 2H); 2.26 (m, I1H); 3.52 (m, 2H); 3.78 (s, 3H); 3.80 (s, 3H); 4.54 (m, I1H); 4.81 (m, 2H); 6.29 (dt, 1H, J=l15.9); 6.80 (s, 2H); 6.95 (d, 1H, J-l15.9); 6.98 (s, 1H). Example 18 is compound 22 in Tables I and III.

### Example 19

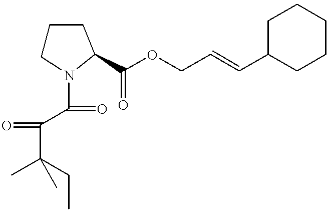

3-Cyclohexyl-1-prop-2(E)-enyl (2S)-1-(3,3-d imethyl-1,2-dioxopentyl )-2-pyrrolidinecarboxylate.

¹H NMR (CDCl₃; 360 MHz): δ0.86 (t, 3H); 1.13-1.40 (m +2 singlets, 9H total); 1.50-1.87 (m, 8H); 1.87-2.44 (m, 6H); 3.34-3.82 (m, 2H), 4.404.76 (m, 3H); 5.35-5.60 (m, 1H); 5.60-5.82 (dd, 1H, J=6.5, 16).

Examples 18 and 19 were prepared according to the synthetic procedure outlined for Examples 4-6, except that the requisite trans-allylic alcohols in the reaction mixture were 3-(2,5-Dichlorophenyl)-1-prop-2(E)-enol and 3-Cyclohexyl-1-prop-2(E)-enol, respectively.

### Example 20

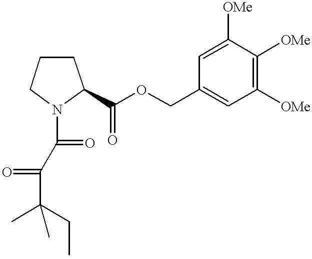

(3,4,5-Trimethoxy)benzyl (2S)-1-(3,3-dimethyl-1,2-dioxopentyl )-2-pyrrolidinecarboxylate.

¹H NMR (CDCl₃; 300 MHz): δ0.85 (t, 3H); 1.20 (s, 3H); 1.22 (s, 3H); 1.58-1.81 (m, 2H); 1.82-2.27 (m, 4H); 3.52 (m, 2H); 3.84 *s, 3H); 3.87 (s, 6H); 4.55 (m, 1H); 5.13 (s, 2H); 6.59 (s, 2H).

### Example 21

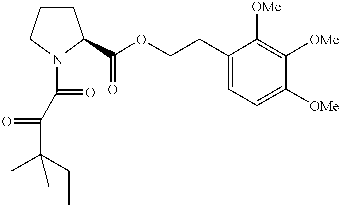

2-(3 ,4,5-Trimethoxyphenyl)-1l-ethyl (2S)-1l-(3 ,3-d imethyl-1 ,2-d ioxopentyl)-2-pyrrolidinecarboxylate

¹H NMR (CDCl₃; 300 MHz): δ0.84 (t, 3H); 1.15 (s, 3H); 1.24 (s, 3H); 1.71 (dm, 2H); 1.98 (m, 5H); 2.24 (m, 1H); 2.63 (m, 2H); 3.51 (t, 2H); 3.79 (s, 3H); 3.83 (s, 3H); 4.14 (m, 2H); 4.52 (m, 1H); 6.36 (s, 2H).

### Example 22

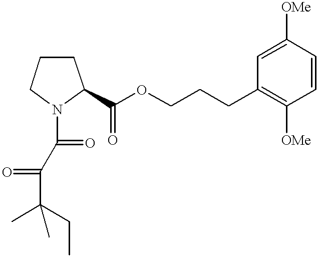

3-(2,5-Dimethoxyphenyl)-1-propyl (2S)-1-(3,3-dimethyl-1,2-dioxopentyl)-2-pyrrolidinecarboxylate.

¹H NMR (CDCl₃; 300 MHz): δ0.87 (t, 3H); 1.22 (s, 3H); 1.26 (s, 3H); 1.69 (m, 2H); 1.96 (m, 5H); 2.24 (m,1H); 2.68 (m, 2H); 3.55 (m, 2H); 3.75 (s, 3H); 3.77 (s, 3H); 4.17 (m, 2H); 4.53 (d, 1H); 6.72 (m, 3H).

Examples 20, 21, and 22 were prepared according to the synthetic procedure outlined for Examples 4-6, except that the requisite di- or trimethoxyphenyl-substituted alcohols in the reaction mixture were (3,4,5-trimethoxy)benzyl alcohol, 2-(3,4,5-trimethoxyphenyl)-1-ethanol, and 3-(2,5-dimethoxyphenyl)-propan-1-ol, respectively.

### Example 23

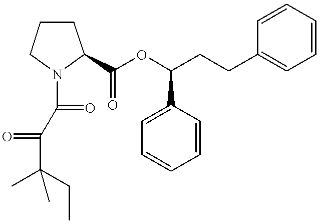

(1S)-1,3-Diphenyl-propyl-(2S)-1-(3,3-dimethyl-1,2-dioxopentyl)-2-(5 pyrrolidinecarboxylate.

¹H NMR (CDCl₃; 360 MHz): δ0.87 (t, 3H); 1.20 (s, 3H); 1.24 (s, 3H); 1.62-2.32 (m, 8H); 2.62-2.75 (m, 2H); 3.43-3.60 (m, 2H); 4.58-4.73 (m, 1H); 5.76 (td,1H, J=1.8, 6.7); 7.19 (m, 3H); 7.24-7.35 (m, 7H).

Example 23 was prepared according to the synthetic procedure outlined for

Examples 4-6, except that the requisite optically active 1-substituted alkanol in the reaction mixture was (1 S)-1,3-diphenylpropan-1-ol.

### Example 24

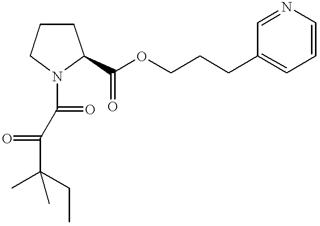

3-(3-Pyridyl )-1-propyl (2S)-1-(3,3-dimethyl-1,2-dioxopentyl)-2-pyrrolidinecarboxylate.

¹H NMR (CDCl₃); 360 MHz): δ0.85 (t, 3H); 1.23,1.26 (s, 3H each); 1.69-1.90 (m, 3H); 1.95-2.01 (m, 4H); 2.20 (m, 1H); 2.72 (t, 2H); 3.53 (m, 2H); 4.18 (m, 2H); 4.52 (m, 1H); 7.22 (m, 1H); 7.53 (dd, 1H); 8.45 (m, 2H).

Example 24 was prepared according to the synthetic procedure outlined for

Examples 4-6, except that the requisite alcohol in the reaction mixture was 3-(3-pyridyl)-propan-1-ol. Example 24 is compound 23 in Tables I and III.

### Example 25

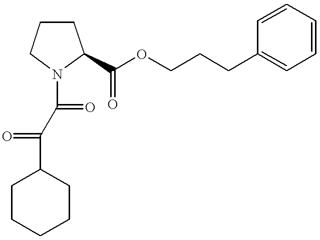

3-Phenyl-1-propyl (2 S)-1-(cyclohexylglyoxyl)-2-pyrrolidinecarboxylate.

¹H NMR (CDCl₃; 300 MHz): δ1.09-1.33 (m, 5H); 1.62-2.33 (m, 12H); 2.69 (t, 2H, J=7.5); 3.15 (dm, 1H); 3.68 (m, 2H); 4.16 (m, 2H); 4.53, 4.84 (d, 1H total); 7.19 (m, 3H); 7.29 (m, 2H). Example 25 is compound 24 in Tables I and III.

### Example 26

The requisite substituted alcohols may be prepared by a number of methods known to those skilled in the art of organic synthesis. As described in Scheme II, alkyl or aryl aldehydes may be homologated to phenyl propanols by reaction with methyl (triphenylphosphoranylidene) acetate to provide a variety of trans-cinnamates; these latter may be reduced to the saturated alcohols by reaction with excess lithium aluminum hydride, or sequentially by reduction of the double bond by catalytic hydrogenation and reduction of the saturated ester by appropriate reducing agents. Alternatively, the trans-cinnamates may be reduced to (E)-allylic alcohols by the use of diisobutylaluminum hydride.

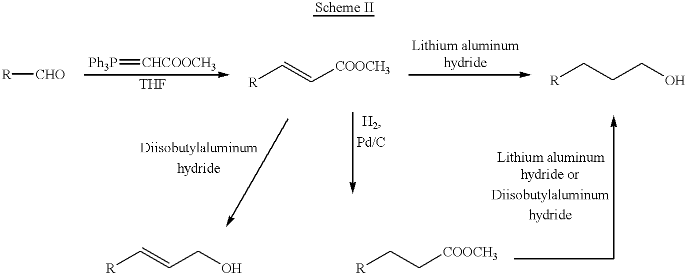

Longer chain alcohols may be prepared by homologation of benzylic and higher aldehydes. Alternatively, these aldehydes may be prepared by conversion of the corresponding phenylacetic and higher acids, and phenethyl and higher alcohols.

### Example 26a

General procedure for the synthesis of acrylic esters, exemplified for methyl (3,4,5-trimethoxy)-trans-cinnamate:

A solution of 3,4,5-trimethoxybenzaldehyde (5.0 g; 25.48 mmol) and methyl (triphenyl-phosphoranylidene)acetate (10.0 g; 29.91 mmol) in tetrahydrofuran (250 mL) was refluxed overnight. After cooling, the reaction mixture was diluted with 200 mL of ethyl acetate and washed with 2×200 mL of water, dried, and concentrated in vacuo. The crude residue was chromatographed on a silica gel column, eluting with 25% ethyl acetate in hexane, to obtain 5.63 g (88%) of the cinnamate as a white crystalline solid, ¹H NMR (300 MHz; CDCl₃): δ3.78 (s, 3H); 3.85 (s, 6H); 6.32 (d, 1H, J=16); 6.72 (s, 2H); 7.59 (d, 1H, J=16).

### Example 26b

Methyl (4,5-dichloro)-trans-cinnamate, 80%, ¹H NMR (300 MHz; CDCl₃): δ3.79 (s, 3H); 6.40 (d, 1H, J=16.8); 7.32 (dd, 1H, J=1.5, 8.1); 7.44 (d, 1H, J=8.1); 7.56 (d, 1H, J=16); 7.58 (s,1H). This compound was prepared by the method of Example 26 from 4,5-dichlorobenzaldehyde.

### Example 26c

Methyl (2-cyclohexyl)-(E)-acrylate, 80%, ¹H NMR (360 MHz; CDCl₃): δ1.12-1.43 (m, 5H); 1.52-1.87 (m, 5H); 2.12 (m, 1H); 2.12 (m, 1H); 3.71 (s, 3H); 5.77 (dd, 1H, J=1.2,15.8); 6.92 (dd, 1H, j=6.8, 15.8). This compound was the precursor for the allylic alcohol used in Example 19.

### Example 26d

General procedure for the synthesis of saturated alcohols from acrylic esters. Exemplified for (3,4,5-trimethoxy)phenylpropanol.

A solution of methyl (3,4,5-trimethoxy)-trans-cinnamate (1.81 g; 7.17 mmol) in tetrahydrofuran (30 mL) was added in a dropwise manner to a solution of lithium aluminum hydride (14 mmol) in THF (35 mL), with stirring and under an argon atmosphere. After the addition was complete, the mixture was heated to 75° C. for 4 hours. After cooling, it was quenched by the careful addition of 15 mL of 2N NaOH followed by 50 mL of water. The resulting mixture was filtered through Celite to remove solids, and the filter cake was washed with ethyl acetate. The combined organic fractions were washed with water, dried, concentrated in vacuo, and purified on a silica gel column, eluting with ethyl acetate to obtain 0.86 g (53%) of the alcohol as a clear oil, ¹H NMR (300 MHz; CDCl₃): δ1.23 (br, 1H); 1.87 (m, 2H); 2.61 (t, 2H, J=7.1); 3.66 (t, 2H); 3.80 (s, 3H); 3.83 (s, 6H); 6.40 (s, 2H).

This compound was a precursor for the compounds synthesized in Examples 8 and 31.

### Example 26e

General procedure for the synthesis of trans-allylic alcohols from acrylic esters. Exemplified for (3,4,5-trimethoxy)phenylprop-2-(E)-enol.

A solution of methyl (3,4,5-trimethoxy)-trans-cinnamate (1.35 g; 5.35 mmol) in toluene (25 mL) was cooled to −10° C. and treated with a solution of diisobutylaluminum hydride in toluene (11.25 mL of a 1.0 M solution; 11.25 mmol). The reaction mixture was stirred for 3 hrs at 0° C. and then quenched with 3 mL of methanol followed by 1 N HCl until the pH was 1. The reaction mixture was extracted into ethyl acetate and the organic phase was washed with water, dried and concentrated. Purification on a silica gel column eluting with 25% ethyl acetate in hexane furnished 0.96 g (80%) of a thick oil, ¹H NMR (360 MHz; CDCl₃): δ3.85 (s, 3H); 3.87 (s, 6H); 4.32 (d, 2H, J=5.6); 6.29 (dt, 1H, J=15.8, 5.7), 6.54 (d, 1H, J=15.8); 6.61 (s, 2H).

This compound was a precursor for the compounds synthesized in Examples 9 and 33.

### Example 26f

(4,5-Dichloro)phenylprop-2-(E)-enol, 89%, ¹H NMR (360 MHz; CDCl₃): δ1.55 (s, 3H); 4.34 (d, 2H, J=4.4); 6.36 (dt, 1H, J=1 5.9, 5.3); 6.54 (d, 1H, J=15.9); 7.20 (dd, 1H, J=8.3, 1.7); 7.38 (d, 1H. J=8.3); 7.45 (d, 1H, J=1.6). This compound was a precursor for the compounds synthesized in Examples 10 and 32.

### Example 26g

Phenylprop-2-(E)-enol, 85%, ¹H NMR (360 MHz; CDCl₃): δ1.72 (br, 1H); 4.31 (d, 2H, J=5.7); 6.36 (dt, 1H, J=15.9, 5.7); 6.61 (d, 1H, J=15.9); 7.02-7.55 (m, 5H). This compound was a precursor for the compounds synthesized in Examples 7 and 34.

### Example 26h

Alcohols containing a substituent at the 1-position of the side chain may be conveniently prepared by addition of appropriate nucleophiles to aldehydes, as described in Scheme III. In cases where optically active substituted alcohols are desired, the racemic alcohols may be oxidized to prochiral ketones and subjected to asymmetric reduction by one of several methods well known to those skilled in the art.

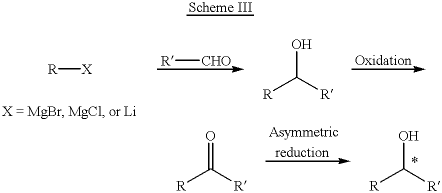

### Example 26i

General procedure for the preparation of 1-substituted alkanols as in Example 26h, exemplified for the synthesis of 1,3-diphenylpropanol.

A solution of 2-(bromoethyl)benzene (17.45 g; 94.3 mmol) in 50 mL of dry diethyl ether was added dropwise, under a nitrogen atmosphere, to a stirred slurry of magnesium turnings (2.50 g; 102.8 mmol) in 50 mL of ether. The mixture was initially heated with a heat gun until reflux had become self-sustaining. After the addition was complete, the mixture was heated externally for 30 min to maintain reflux. A solution of 10.01 g (94.3 mmol) of benzaldehyde in 20 mL of ether was then added dropwise, and reflux was continued for 30 min. After cooling, the reaction mixture was poured into 150 mL of saturated ammonium chloride and extracted into ethyl acetate. The crude material obtained upon removal of the solvent was purified on a flash column, eluting with 5% ethyl acetate/hexane to 20% ethyl acetate, to obtain 13.73 g (69%) of the alkanol as a light yellow oil, ¹H NMR (360 MHz; CDCl₃): δ1.93-2.30 (m, 3H); 2.70-2.90 (m, 2H); 4.72 (br, 1H); 7.19-7.27 (m, 3H); 7.27-7.36 (m, 3H); 7.36-7.47 (m, 4H).

This compound was a precursor for the compounds synthesized in Examples 11 and 23.

### Example 26i

General procedure for conversion of racemic 1-substituted alkanols, for example, from Example 35, to optically active 1-substituted alkanols via prochiral ketones. Exemplified for (1R)-1,3-diphenyl-1-propanol.

A solution of racemic 1,3-diphenyl-1-propanol (1,26 g; 5.94 mmol) was dissolved in 10 mL of acetone, and Jones reagent was added until persistence of the orange color. After stirring for 30 min, the reaction was quenched by adding 2 mL of 2-propanol. The solvent was decanted away from the precipitated solids, which were washed with ethyl actetate. The combined organic fractions were washed with 2×20 mL of water, dried and concentrated. The crude product was filtered through a plug of silica gel, eluting with 25% ethyl acetate/hexane, to obtain 1.07 g (86%) of 1,3-diphenylpropanone as a white crystalline solid, ¹H NMR (360 MHz; CDCl₃): δ3.09 (t, 2H, J=8.1); 3.33 (t, 2H, J=8.1); 7.29 (m, 5H); 7.49 (m, 3H); 7.98 (m, 2H).

A solution of 1,3-diphenylpropanone (1.07 g; 5.09 mmol) in tetrahydrofuran (10 mL) was cooled to −23° C. and treated with an asymmetric reducing agent, (+)-B-chlorodiisopinocampheyl-borane (1.80 g; 5.60 mmol) in 20 mL THF, and the resulting solution was allowed to stand overnight at −23° C. After evaporating to dryness, the residue was treated with ether (65 mL) and diethanolamine (1.0 g) and stirred for 3 hrs. The mixture was then filtered to remove solids and concentrated, and the residue was purified using gradient elution (5% ethyl acetateihexane to 10% ethyl acetate) on a silica gel column to obtain 660 mg (61%) of (1R)-1 ,3-diphenyl-1-propanol as a crystalline white solid, ¹H NMR (360 MHz; CDCl₃): δ1.95-2.15 (m, 3H); 2.59-2.78 (m, 2H); 4.65 (dd, 1H, J=5.4, 7.8); 7.14-7.35 (m, 1OH).

This compound was a precursor for the compound synthesized in Example 1.

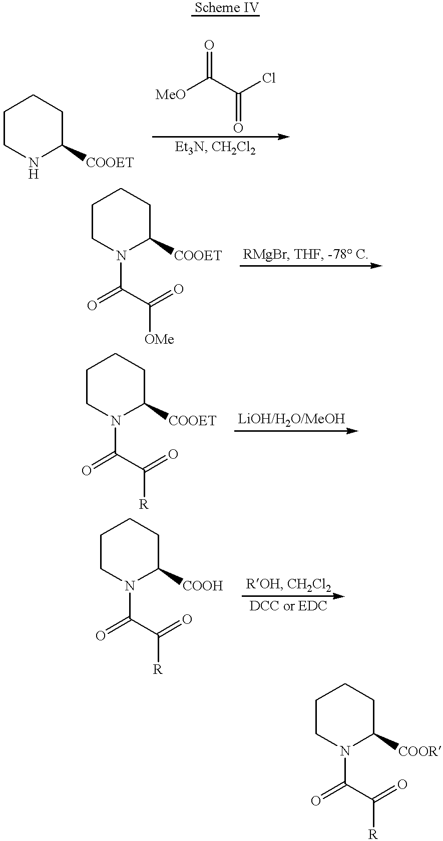

Examples 27 to 42 were synthesized according to Scheme IV.

### Example 27

Synthesis of ethyl 1-(1,2-dioxo-2-methoxyethyl)-2-piperidinecarboxylate.

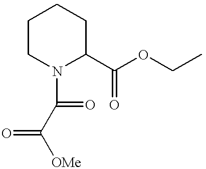

A solution of ethyl pipecolinate (1.00 g; 5.57 mmol) in dry methylene chloride (15 mL) was cooled to 0° C. and treated with triethylamine (1.24 g; 12.25 mmol; 2.1 eq). After stirring the formed slurry under a nitrogen atmosphere for 15 min, a solution of methyl oxalyl chloride (0.96 g; 6.13 mmol) in methylene chloride (15 mL) was added dropwise. The resulting mixture was stirred at 0° C. for 1.5 hr. After filtering to remove solids, the organic phase was washed with water, dried over MgSO₄ and concentrated. The crude residue was purified on a silica gel column, eluting with 50% ethyl acetate in hexane, to obtain 1.21 g (95%) of the product as a reddish oil. Mixture of cis-trans amide rotamers; data for trans rotamer given. ¹H NMR (300 MHz, CDCl₃): δ1.25 (t, 3H); 1.30-1.75 (m, 5H); 2.33 (m, 1H); 3.42 (dt, 1H); 3.57 (br d, 1H); 3.85 (5, 3H); 4.29 (dd, 2H); 5.23 (d,1H).

### Example 28

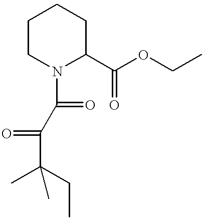

Synthesis of ethyl 1-(1,2-dioxo-3,3-dimethylpentyl)-2-piperidinecarboxylate.

A solution of ethyl 1-(1,2-dioxo-2-methoxyethyl)-2-piperidinecarboxylate (1.43 g; 5.88 mmol) in 20 mL of tetrahydrofuran (THF) was cooled to -78° C. and treated with 6 mL of a 1.0 M solution of 1,1-dimethylpropylmagnesium chloride in THF. After stirring the resulting homogeneous mixture at −78° C. for three hours, the mixture was poured into saturated ammonium chloride (30 mL) and extracted into ethyl acetate. The organic phase was washed with water, dried, and concentrated, and the crude material obtained upon removal of the solvent was purified on a silica gel column, eluting with 25% ethyl acetate in hexane, to obtain 1.35 g (76%) of the oxamate as a colorless oil. ¹H NMR (300 MHz, CDCl₃): δ0.91 (t, 3H); 1.20,1.25 (s, 3H each); 1.30 (t, 3H); 1.35-1.80 (m, 7H); 2.35 (br d,1H); 3.20 (td,1H); 3.41 (br d,1H); 4.20 (q, 2H); 5.22 (d,1H).

### Example 29

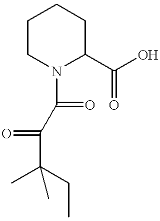

Synthesis of 1-(1,2-dioxo-3,3-dimethylpentyl)-2-piperidinecarboxylic acid

A mixture of ethyl 1-(1,2-dioxo-3,3-dimethylpentyl)-2-piperidinecarboxylate (0.69 g; 2.43 mmol), 1 N LiOH (5 mL), and methanol (20 mL) was stirred at 0° C. for 30 min and at room temperature overnight. The mixture was acidified to pH 1 with 1 N HCl, diluted with water, and extracted into 50 mL of methylene chloride. The organic extract was washed with brine and concentrated to deliver 0.61 g (98%) of snow-white solid which did not require further purification. ¹H NMR (CDCl₃, 300 MHz): δ0.89 (t, 3H, J=7.5); 1.20,1.23 (s, 3H each); 1.65-1.78 (m, 7H); 2.34 (m,1H); 3.40 (m,1H); 3.76 (m, 1H); 4.28 (dd,1H); 10.51 (bs,1H).

### Example 30

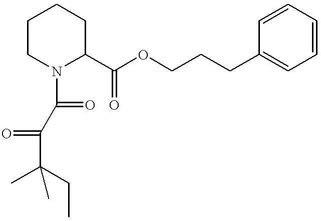

Synthesis of 3-phenyl-1-propyl 1-(1,2-dioxo-3,3-dimethylpentyl)-2-piperidinecarboxylate

A mixture of 1-(1,2-dioxo-3,3-dimethylpentyl)-2-piperidinecarboxylic acid (590 mg; 2.31 mmol), 3-phenylpropanol (520 mg; 3.71 mmol), dicyclohexylcarbodiimide (815 mg; 3.95 mmol), camphorsulphonic acid (180 mg; 0.77 mmol) and 4-dimethyl aminopyridine (95 mg; 0.77 mmol) in methylene chloride (15 mL) was stirred overnight under a nitrogen atmosphere. The reaction mixture was filtered through Celite to remove solids and concentrated in vacuo. The crude material was triturated with several portions of ether, and the ether portions were filtered through Celite to remove solids and concentrated in vacuo. The concentrated filtrate was purified on a flash column (20% ethyl acetate in hexane) to obtain 800 mg (93%) of the product as an oil. ¹H NMR (CDCl₃, 300 MHz): δ0.85 (t, 3H); 1.23,1.26 (s, 3H each); 1.63-1.94 (m, 9H); 2.32 (m, 1H); 2.69 (m, 2H); 3.21 (m, 1H); 3.35 (m, 1H); 4.17 (m, 2H); 5.24 (m, 1H); 7.14 (m, 3H); 7.7.23 (m, 2H).

### Example 31

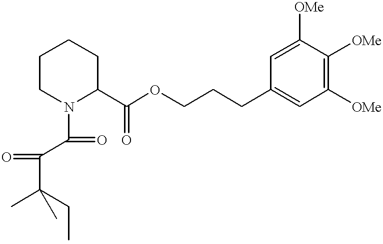

3-(3,4,5-Trimethoxyphenyl)-1-propyl 1-(3,3-d i methyl-1,2-d ioxopentyl)-2-piperidinecarboxylate: ¹H NMR (CDCl₃, 300 MHz): δ0.80 (t, 3H); 1.18 (s, 6H); 1.67 (m, 7H); 1.94 (m, 2H); 2.29 (brd, 1H); 2.61 (t, 2H); 3.17 (td, 1H); 3.35 (d, 1H); 3.79 (s, 3H); 3.81 (s, 6H); 4.15 (m, 2H); 5.24 (d, 1H).

### Example 32

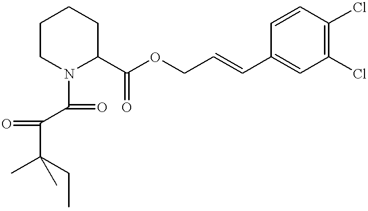

3-(4,5-Dichlorophenyl)-1-prop-2-(E)-enyl 1-(3,3-dimethyl-1,2-dioxopentyl)-2-piperidinecarboxylate: ¹H NMR (CDCl₃, 360 MHz): δ0.89 (t, 3H); 1.18 (s, 3H); 1.24 (s, 3H); 1.57-1.89 (m, 7H); 2.38 (d,1H); 3.20-3.28 (dt, 1H); 3.30-3.43 (dm,1H); 4.81 (d, 2H); 5.31 (d, 1H); 6.16-6.36 (m, 1H); 6.48-6.68 (d, 1H); 7.20 (d, 1H); 7.39 (d, 1H); 7.47 (s, 1H).

### Example 33

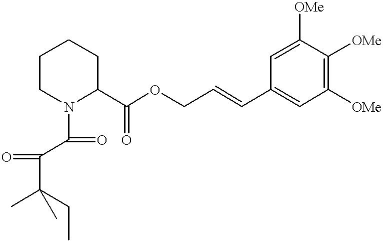

3-(3,4,5-Trimethoxyphenyl)-1-prop-2-(E)-enyl 1-(3,3-dimethyl-1,2-dioxopentyl)-2-piperidinecarboxylate: ¹H NMR (CDCl₃, 360 MHz): δ0.89 (t, 3H); 1.21 (s, 3H); 1.24 (s, 3H); 1.41-1.85 (m, 7H); 2.35 (d,1H); 3.25 (t,1H); 3.39 (m,1H); 3.86 (s, 3H); 3.89 (s, 6H); 4.81 (m, 2H); 5.33 (d,1H); 6.21 (m,1H); 6.61 (d,1H); 6.63 (s, 2H).

### Example 34

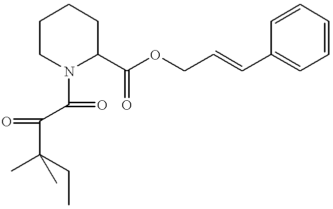

3-Phenyl-1-prop-2-(E)-enyl 1-(3,3-dimethyl-1,2-dioxopentyl)-2-piperidinecarboxylate: ¹H NMR (CDCl₃, 360 MHz): δ0.88 (t, 3H); 1.20 (s, 3H); 1.24 (s, 3H); 1.25-1.77 (m, 6H); 1.86-2.06 (m, 1H); 2.30-2.40 (m, 1H): 3.24 (t, 1H); 3.41 (d, 1H); 4.82 (d,1H); 5.31 (d, 1H); 6.25-6.29 (m,1H); 6.68 (d, 1H); 7.26-7.54 (m, 5H).

### Example 35

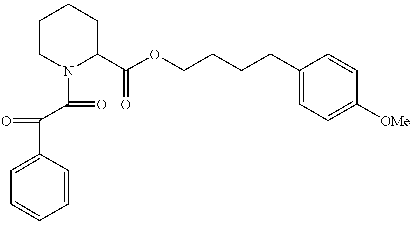

4-(4-Methoxyphenyl)butyl N-(phenylglyoxyl) )-2-piperidinecarboxylate: ¹H NMR (CDCl₃, 300 MHz): δ1.26-1.78 (m, 9H); 2.36 (d, 1H); 2.58 (m, 2H); 3.25 (m, 1H); 3.48 (dm, 1H); 3.78 (s, 3H); 4.24 (m, 2H); 5.40 (m, 1H); 6.82 (d, 2H); 7.09 (d, 2H); 7.64 (m, 2H); 7.66 (m, 1H); 8.02 (m, 2H). Anal. Calcd. for C₂₅H₂₉NO₅: C, 70.90; H, 6.90; N, 3.31. Found: C, 70.87; H, 6.92; N, 3.36. Example 35 is compound 3 in Tables I and III.

### Example 36

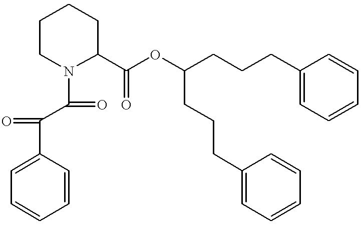

1,7-Diphenylheptyl N-(phenylglyoxyl) )-2-piperidinecarboxylate: ¹H NMR (CDCl₃, 300 MHz): δ1.21-1.85 (m, 14H); 2.48 (m, 4H); 3.22 (m, 1H); 3.44 (m, 1H); 5.09 (br, 1H); 5.38 (br, 1H); 7.06-8.04 (m, 15H). Anal. Calcd. for C₃₃H₃₇NO₄: C, 77.47; H, 7.29; N, 2.74. Found: C, 77.39; H, 7.32; N, 2.66. Example 36 is compound 20 in Tables I and III.

### Example 37

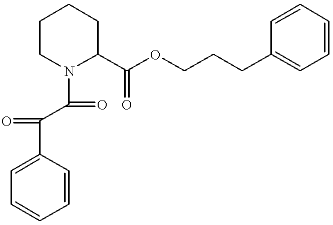

3-Phenyl-1-propyl N-(phenylglyoxyl)-2-piperidinecarboxylate: ¹H NMR (CDCl₃, 300 MHz): δ1.36-2.05 (m, 7H); 2.36 (d, 1H); 2.74 (m, 2H); 3.24 (t, 1H); 3.50 (t, 1H); 4.25 (m, 2H); 5.42 (m, 1H); 7.28 (m, 4H); 7.64 (m, 4H); 8.03 (m, 2H). Anal. Calcd. for C₂₃H₂₅NO₄: C, 72.80; H, 6.64; N, 3.69. Found: C, 72.74; H, 6.62; N, 3.62. Example 37 is compound 2 in Tables I and III.

### Example 38

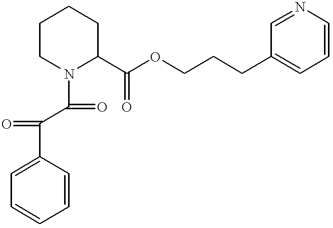

3-(3-Pyridyl)-1-propyl N-(phenylglyoxyl)-2-piperidinecarboxylate: ¹H NMR (CDCl₃, 300 MHz): δ1.26-2.08 (m, 7H); 2.35 (d,1H); 2.75 (t, 2H); 3.29 (t,1H); 3.49 (d, 1H); 4.27 (t, 2H); 5.42 (d, 1H); 7.23 (m,1H); 7.52 (m, 3H); 7.63 (m,1H); 8.03 (m, 2H); 8.48 (m, 2H). Anal. Calcd. for C₂₂H₂₄N₂O₄: 0.25 H₂O₄: C, 68.64; H, 6.42; N, 7.28. Found: C, 68.37; H, 6.41; N, 7.22.

### Example 39

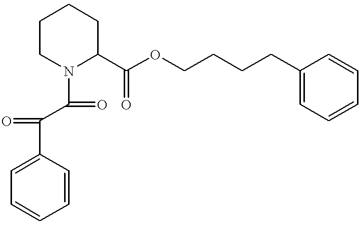

4-Phenyl-1-butyl N-(phenylglyoxyl)-2-piperidinecarboxylate: ¹H NMR (CDCl₃, 300 MHz): δ1.26-1.80 (m, 12H); 2.67 (m, 2H); 3.23 (t, 1H); 3.49 (t, 1H); 4.25 (m, 2H); 5.40 (m, 1H); 7.18 (m, 3H); 7.26 (m, 2H); 7.48 (m, 2H); 7.64 (m, 1H); 8.03 (m, 2H). Anal. Calcd. for C₂₄H₂₇NO₄: C, 73.26; H, 6.92; N, 3.56. Found: C, 73.19; H, 6.94; N, 3.64. Example 39 is compound 4 in Tables I and III.

### Example 40

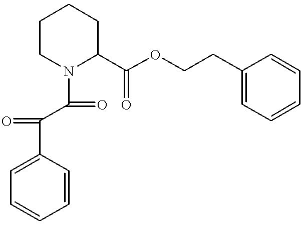

2-Phenyl-1-ethyl N-(phenylglyoxyl)-2-piperidinecarboxylate: ¹H NMR (CDCl₃, 300 MHz): δ1.23-1.75 (m, 5H); 2.21 (d, 1H); 3.09 (m, 3H); 3.41 (d, 1H); 4.48 (m, 2H); 5.38 (m, 1H); 7.27 (m, 5H); 7.53 (m, 2H); 7.65 (m, 1H); 8.01 (m, 2H). Anal. Calcd. for C₂₂H₂₃NO₄: 0.25H₂O: C, 71.43; H, 6.40; N, 3.79. Found: C, 71.60; H, 6.50; N, 4.12.

### Example 40 is compound 5 in Tables I and III.

### Example 41

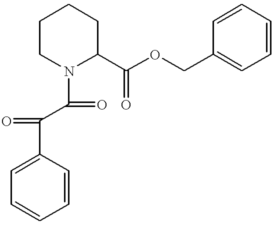

Benzyl N-(phenylglyoxyl)-2-piperidinecarboxylate: ¹H NMR (CDCl₃, 300 MHz): 6 1.38-1.81 (m, 5H); 2.41 (d, 1H); 3.22 (m, 1H); 3.48 (d, 1H); 5.26 (s, 2H); 5.47 (d, 1H); 7.42 (m, 7H); 7.61 (m, 1H); 7.97 (m, 2H). Anal. Calcd. for C₂, H₂₁NO₄-0.25H₂O: C, 71.78; H, 6.02; N, 3.99. Found: C, 71.90; H, 6.12; N, 4.01. Example 41 is compound 1 in Tables I and III.

### Example 42

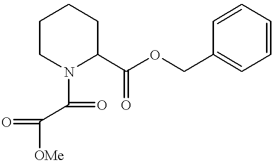

Benzyl N-(methoxyglyoxyl)-2-piperidinecarboxylate: ¹H NMR (CDCl₃, 300 MHz): δ1.26-1.77 (m, 5H); 2.32 (m, 1H); 3.33 (t, I1H); 3.54 (d, 1H); 3.88 (s, 3H); 5.23 (s, 2H); 5.45 (m, 1H); 7.36 (s, 5H). Anal. Calcd. for C₁₆H,gNO₅: C, 62.94; H, 6.27; N, 4.59. Found: C, 62.80; H, 6.35; N, 4.53.

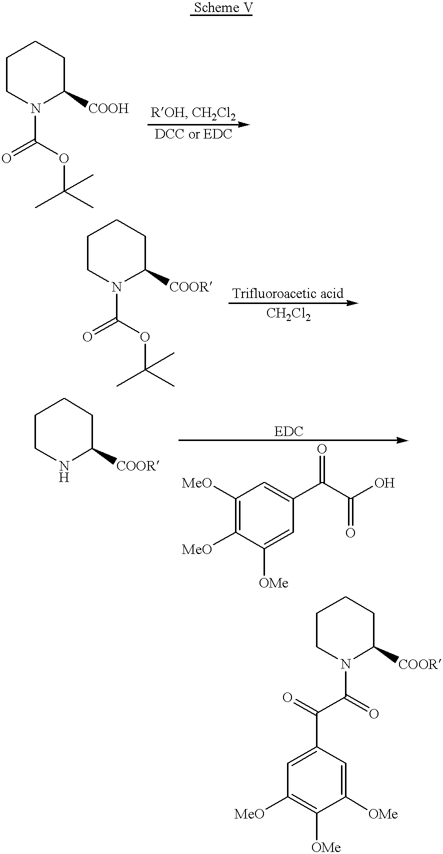

Examples 43 to 50 were synthesized according to Scheme V.

### Example 43

Synthesis of (S)-boc-pipecolyl-1,7-diphenyl-4-heptanyl ester:

A solution of (S)-boc-pipecolic acid (330 mg; 1.44 mmol) in CH₂Cl₂ (20 mL) was treated with 1,7-diphenyl-4-heptanol (350 mg; 1.30 mmol), 1-(3-dimethylaminopropyl)-3-ethylcarbodiimide hydrochloride (280 mg; 1.44 mmol), and a catalytic amount of N,N,-dimethylaminopyridine. The reaction mixture was stirred overnight at room temperature, concentrated, and purified on a silica gel column eluting with 25% ethyl acetate in hexanes to provide 160 mg of product as a clear oil.

(S)-1,7-Diphenyl-4-heptanylpipecolate: A solution of (S)-boc-pipecolyl-1,7-diphenyl-4-heptanyl ester (150 mg) in 10 ml of CH₂Cl₂ was treated with 3 mL of trifluoroacetic acid and stirred at room temperature for 2 hours. It was neutralized with aqueous potassium carbonate and the layers were separated. The organic phase was dried over MgSO₄ and concentrated to provide 70 mg of the free amine.

### Example 44

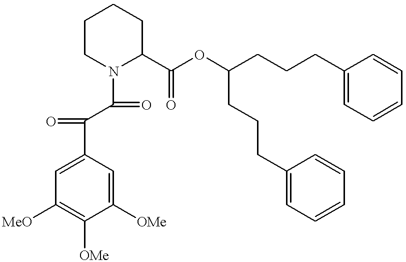

1,7-Diphenyl-4-heptyl (S)-N-(3,4,5-trimethoxyphenylglyoxyl)pipecolate: A solution of (S)-1,7-diphenyl-4-heptanylpipecolate (50 mg; 0.13 mmol) and 3,4,5-trimethoxybenzoyl-formic acid (45 mg; 0.2 mmol) was treated with 1-(3-dimethylaminopropyl)-3-ethyl- carbodiimide hydrochloride (40 mg; 0.2 mmol) and a catalytic amount of N,N,-dimethylaminopyridine. The reaction mixture was stirred overnight at room temperature, concentrated, and purified on a silica gel column eluting with 25% ethyl acetate in hexanes to provide 20 mg of product as a clear oil, ¹H NMR (300 MHz; CDCl₃): δ1.31-1.92 (m, 13H); 2.35 (m, 1H); 2.66 (m, 4H); 3.29 (td, 1H); 3.94 (s, 9H); 5.08 (m, 1H); 5.41 (d, 1H); 7.19 (m, 6H); 7.28 (m, 4H); 7.42 (m, 2H). Example 44 is compound 21 in Tables I and III.

### Example 45

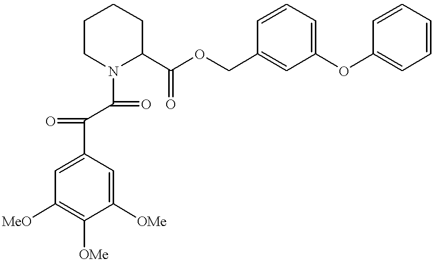

3-(Phenoxybenzyl) (S)-N-(3,4,5-trimethoxyphenylglyoxyl)pipecolate: ¹H NMR (300 MHz; CDCl₃): δ1.22-1.47 (m, 1H); 1.50-1.70 (m, 2H); 1.72-1.93 (m, 2H); 2.39 (d, 1H); 3.25 (td, 1H); 3.51 (d, 1H); 3.96 (s, 9H); 5.18 (m, 2H); 5.43 (d, 1H); 7.01 (m, 4H);

7.15 (m, 3H); 7.37 (m, 4H). Example 45 is compound 13 in Tables I and III.

### Example 46

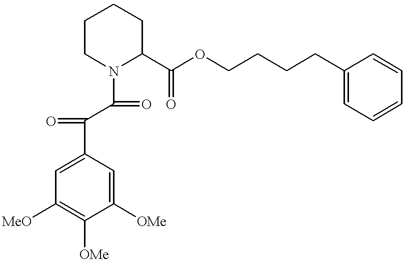

4-Phenylbutyl (S)-N-(3,4,5-trimethoxyphenylglyoxyl)pipecolate: ¹H NMR (300 MHz; CDCl₃): δ1.32-1.88 (m, 9H); 2.35 (d, 1H); 2.63 (m, 2H); 3.25 (td, 1H); 3.48 (d, 1H); 3.93 (s, 9H); 4.18 (m, 2H); 5.35 (d, 1H); 7.17 (m, 3H); 7.23 (m, 2H); 7.36 (s, 2H).

### Example 46 is compound 14 in Tables I and III.

### Example 47

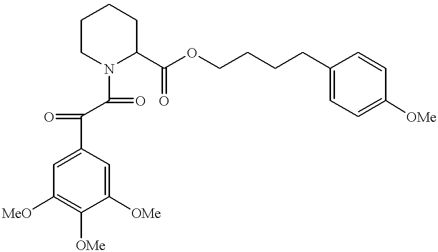

4-(4-Methoxyphenyl)butyl (S)-N-(3,4,5-trimethoxyphenylglyoxyl)pipecolate: ¹H NMR (300 MHz; CDCl₃): 5 1.21-1.92 (m, 9H); 2.37 (m,1H); 2.62 (m, 2H); 3.25 (td,1H); 3.49 (d, 1H); 3.78 (s, 3H); 3.93 (s, 9H); 4.15-4.23 (m, 2H); 5.38 (m, 1H); 6.39 (m, 2H); 7.07 (m, 1H); 7.36 (m, 1H). Example 47 is compound 16 in Tables I and III.

### Example 48

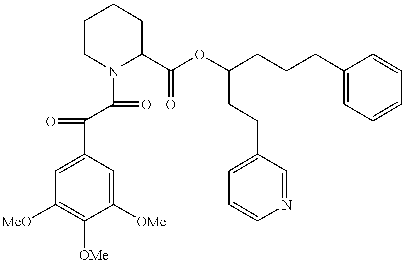

1-Phenyl-6-(3-pyridyl)-4-hexyl (S)-N-(3,4,5-trimethoxyphenylglyoxyl)pipecolate: ¹H NMR (300 MHz; CDCl₃): 61.22-2.01 (m, 11H); 2.39 (m, 1H); 2.65 (m, 4H); 3.32 (m, 1H); 3.53 (m, 1H); 3.92 (s, 9H); 5.06 (m, 1H); 5.40 (dd, 1H); 7.17-7.32 (m, 6H); 7.37 (d, 2H); 7.50 (m, 1H); 8.48 (m, 2H). Example 48 is compound 22 in Tables I and l1l.

### Example 49

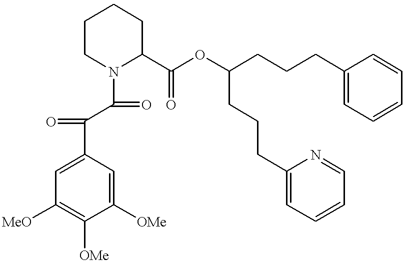

1-Phenyl-7-(2-pyridyl)-4-heptyl (S)-N-(3,4,S-trimethoxyphenylglyoxyl)pipecolate: ¹H NMR (300 MHz; CDCl₃): δ1.23-2.02 (m, 13H); 2.39 (d, 1H); 2.65 (m, 2H); 2.86 (t, 2H); 3.31 (t, 1H); 3.51 (d, 1H); 3.94 (s, 9H); 5.1 0 (m, 1H); 5.40 (m, 1H); 7.16-7.32 (m, 9H); 7.61 (7, 1 H); 8.51 (m, 1H). Example 49 is compound 23 in Tables I and III.

### Example 50

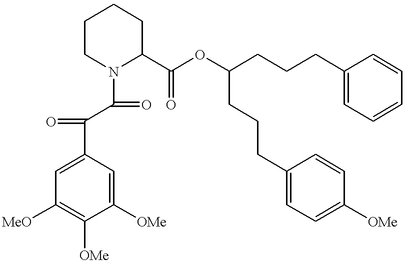

1-Phenyl-7-(4-methoxyphenyl)-4-heptyl (S)-N-(3,4,5-trimethoxyphenylglyoxyl)-pipecolate: ¹H NMR (300 MHz; CDCl₃): δ1.22-1.88 (m, 13H); 2.32 (d, 1H); 2.60 (m, 4H); 3.25 (td,1H); 3.48 (d,1H); 3.76 (s, 3H); 3.91 (s, 9H); 5.05 (m, 1H); 5.37 (m, 1H); 6.80 (d, 2H); 7.00-7.11 (m, 2H); 7.13-7.20 (m, 3H); 7.21-7.28 (m, 2H); 7.47 (s, 2H). 5 Example 50 is compound 24 in Tables I and III.

### Example 51

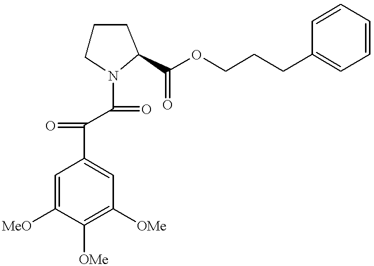

3-Phenyl-1-propyl (2S)-1l-(3,4,5-trimethoxyphenylglyoxyl) pyrrolidinecarboxylate was synthesized according to Scheme V substituting boc-proline for boc pipecolic acid. ¹H NMR (CDCl₃; 300 MHz): δ1.89-2.16 (m, 5H); 2.33 (m, 1H); 2.72 (m, 2H); 3.65 (m, 2H), 3.85, 3.86, 3.88 (s, 9H total); 4.21 (m, 2H); 4.66 (m, 1H); 6.80 (s, 2H); 7.18-7.32 (m, 5H).

### Example 52

Synthesis of piperidine ketone compounds

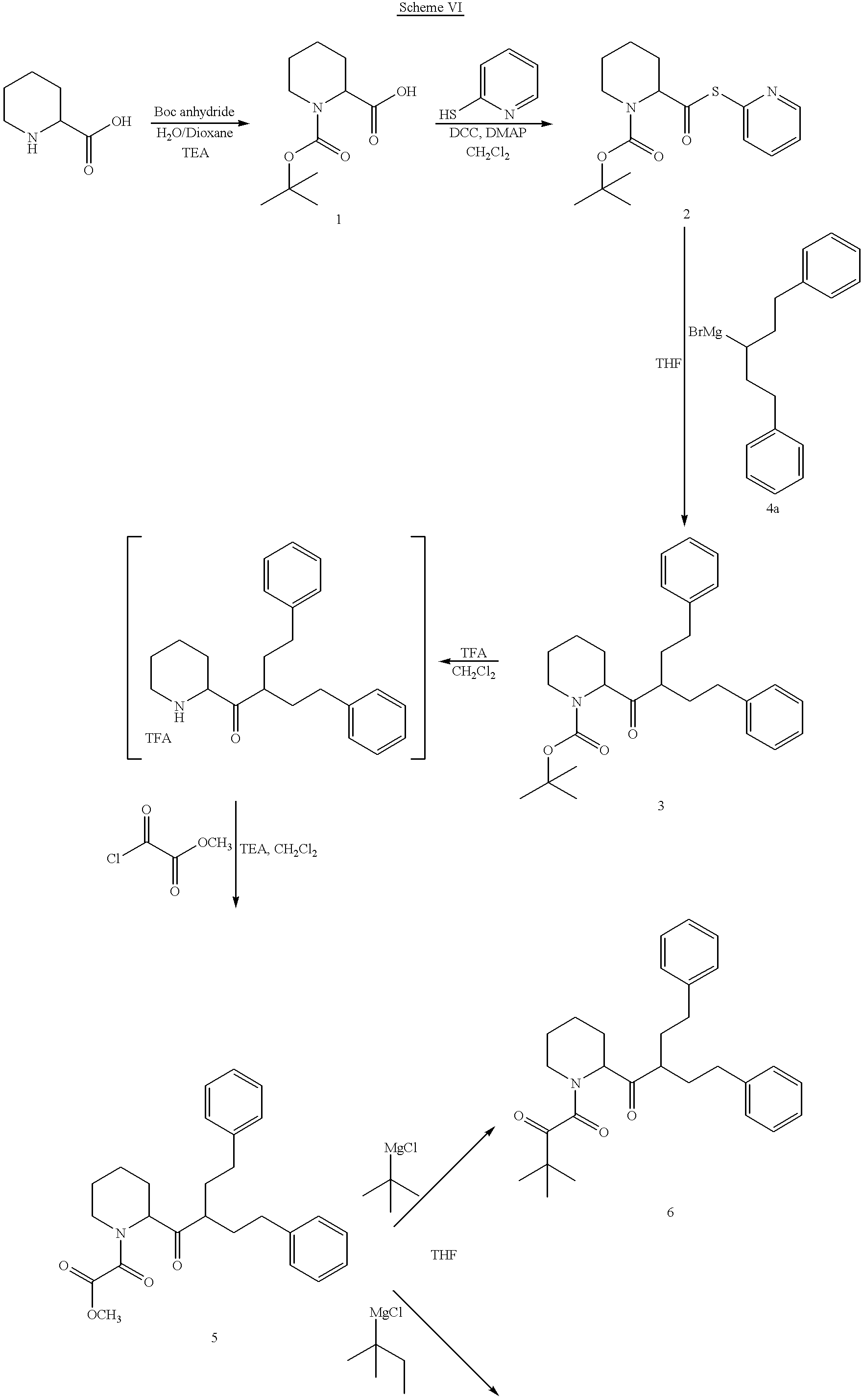

Piperidine-1,2-dicarboxylic acid 1-tert-butyl ester (1). To a solution of 10.0 g (77.42 mmol) of piperidine-2-carboxylic acid in H₂O/Dioxane (2OOmL, 1:1) was added 18.59g (85.16 mmol) of di-tert-butyl dicarbonate followed by 8.62g (85.16 mmol) of triethylamine, and the mixture was stirred for 16 hrs at ambient temperature. The solution was evaporated to remove excess dioxane, diluted with H₂O (100 mL) and extracted with CH₂Cl₂ (2×200 mL). The organic phase was dried (MgSO₄) and the evaporated to give a yellow oil which was subjected to column chromatography (CHCl₃/MeOH/AcOH, 9:.8:.2) to yield 12.9g (73.0%) of 1 as a yellow oil. TLC R_{f}=0.7 (CHCl₃/MeOH/AcOH, 9:.8:.2)

2-(Pyridin-2-ylsulfanylcarbonyl)-piperidine-1-carboxylic acid tert-butyl ester (2).

To a solution of 12.9g (56.51 mmol) of 1 and 17.49g (84.77 mmol) of 1,3-dicyclohexylcarbodiimide in CH₂Cl₂ (250 mL) was added 9.42g (84.77 mmol) of pyridine-2-thiol followed by 0.25g (0.2 mmol) of 4-dimethylaminopyridine, and the mixture was stirred for 16 hrs at ambient temperature. The slurry was filtered and the resulting organic phase was evaporated to yield a yellow oil which was subjected to column chromatography (EtOAc/Hexanes, 3:2) to yield 11.2g (61.5%) of 2 as a yellow oil. TLC R_{f}=0.6 (EtOAc/Hexanes, 3:2)

2-(2-Phenethyl-4-phenyl-butyryl)-piperidine-1-carboxylic acid teff-butyl ester (3).

To a solution of 0.95g (2.95 mmol) of 2 in anhydrous THF (12 mL) was added dropwise at 0° C. a solution of 5.4 mL (3.24 mmol) of 4a over 5 minutes. After 2 hours at 0° C., the solution was allowed to warm to ambient temperature for 18 hrs. The solution was quenched with H₂O and extracted with ether (3×50 mL). The organic phase was washed with brine, dried (MgSO₄) and evaporated to a clear oil which was subjected to column chromatography (EtOAc/Hexanes, .5:9.5) to yield 0.24g (18.7%) of 3 as a clear oil. TLC R_{f}=0.4 (EtOAc/Hexanes, .5:9.5)

Oxo-[2-(2-phenethyl-4-phenyl-butyryl)-piperidin-1-yl]-acetic acid methyl ester (5). To a solution of 0.24g (0.55 mmol) of 3 in CH₂Cl₂ (2 mL) was added dropwise 0.13 mL (1.65 mmol) of trifluoroacetic acid, and the mixture was allowed to stir for 2 hours. The solution was diluted with CH₂Cl₂ (10 mL) and cooled to 0° C. followed by dropwise addition of 0.3g (3.00 mmol) of triethylamine. After 5 minutes, to the solution was added dropwise 0.08g (0.61 mmol) of chlorooxoacetate, and the mixture was stirred for 2 hours. The solution was quenched with H₂O and extracted with CH₂Cl₂ (2×200 mL). The organic phase was dried (MgSO₄) and evaporated to a clear oil which was subjected to column chromatography (EtOAc/Hexanes, 1:3) to yield 0.19g (81.9%) of 5 as a clear oil. TLC R_{f}=0.5 (EtOAc/Hexanes, 1:3)

3,3-Dimethyl-1-[2-(2-phenethyl-4-phenyl-butyryl)-piperidin-1-yl]-butane-1,2-dione

(6). To a solution of 0.16g (0.38 mmol) of 5 in anhydrous THF (2 mL) was added dropwise at −78° C. a 2.0 M solution of 0.21 mL (0.42 mmol) of tert-butyl magnesium chloride in THF, and the mixture was stirred for 3 hours at −78° C. The solution was poured over saturated ammonium chloride (50 mL) and extracted with EtOAc (3×100 mL). The organic phase was dried (MgSO₄) and evaporated to a clear oil which was subjected to column chromatography (EtOAc/Hexanes, 1:4) to yield 0.13g (76.5%) of 6 as a clear oil. TLC R_{f}=0.62 (EtOAc/Hexanes, 1:4) ¹H NMR (CDCl₃, 300 MHz): 5 1.40 (s, 9H); 1.36-2.29 (m, 9H); 2.62-2.82 (m, 4H); 3.39-3.52 (m, 2H); 5.18 (m, 1H); 5.31 (d, 1H, J=6.2); 5.42 (d, 1H, J=5.2); 7.42-7.26 (m, 10H); Anal. (C₂₉H₃₇NO₃) C, H, N.

3,3-Dimethyl-1-[2-(2-phenethyl-4-phenyl-butyryl)-piperid in-1-yl]-pentane-1 ,2-dione (7). To a solution of 0.38g (0.90 mmol) of 5 in anhydrous THF (5 mL) was added dropwise at −78° C. a 1.0M solution of 1.9 mL (1.90 mmol) of 1 ,1-dimethylpropyl magnesium chloride in THF, and the mixture was stirred for 3 hours at −78° C. The solution was poured over saturated ammonium chloride (50 mL) and extracted with EtOAc (2×100 mL). The organic phase was dried (MgSO₄) and evaporated to a clear oil which was subjected to column chromatography (EtOAc/Hexanes, 1:4) to yield 0.31g (74.5%) of 6 as a clear oil. TLC R_{f}=0.8 (EtOAc/Hexanes, 1:4) ¹H NMR (CDCl₃, 400 MHz): δ0.94 (t, 3H, J=7.5 Hz); 1.24 (s, 3H); 1.28 (s, 3H); 1.42-2.06 (m,₁ 1H); 2.36 (d, 1H, J=13.OHz); 2.61-2.89 (dt, 1H, J=3.2,12.9 Hz); 3.42 (brt, 1H, J=12.8 Hz); 5.08 (ddd, 1H, J=5.2,7.2,12.3); 5.32 (d, 1H, J=5.4 Hz); 7.16-7.32 (m, 10H); Anal. (C₃₀H₃₉NO₃) C, H, N.

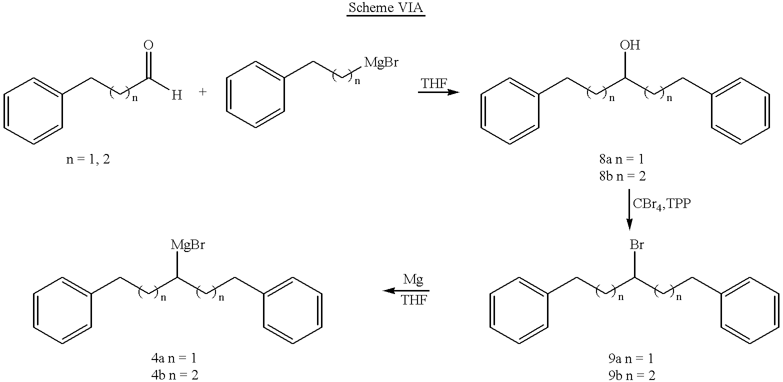

Common Intermediates:

1,5-Diphenyl-pentan-3-ol (8a).

To a solution of 12.8g (95.2 mmol) of 3-phenyl-propionaldehyde in anhydrous THF (100 mL) was added dropwise at 0° C. a 1.0 M solution of 100 mL (100 mmol) of phenethyl magnesium bromide in THF, and the mixture was stirred for 2 hours at 0° C. The solution was poured over saturated ammonium chloride and extracted with ether (3×1 50 mL). The organic phase was dried (MgSO₄) and evaporated to a solid, which was subject to column chromatography (EtOAc/Hexanes, 1:9) to yield 1 0.0g (22.8%) of 8a as a white solid. TLC R_{f}=0.5 (EtOAc/Hexanes, 1:9)

3-Bromo-1,5-diphenylpentane (9a).

To a solution of 2.29g (95.3 mmol) of 8a and 3.48g (10.48 mmol) of carbon tetrabromide in anhydrous CH₂Cl₂ (80 mL) was added portionwise at 0° C., 2.75g (10.48 mmol) of triphenylphosphine, and the mixture was stirred for 1 hour at 0° C. followed by warming to ambient temperature for 16 hours. The solution was evaporated and redissolved in EtOAc. White solid was filtered and the resulting solution was evaporated to an orange oil which was subject to column chromatography (EtOAc/Hexanes, 1:9) to yield 1.91 g (62.6%) of 9a as a clear oil. TLC R_{f}=0.8 (EtOAc/Hexanes, 1:9)

1,5-Diphenylpentylmagnesium bromide (4a).

A solution of 1.91g (6.30 mmol) of 9a in anhydrous THF (10 mL) was added dropwise to 0.17g (6.93 mmol) of magnesium powder stirred under an inert atmosphere for 16 hours. Upon complete addition, the solution was refluxed at 90° C. for 3 hours. The solution was cooled to an ambient temperature and used directly.

Examples 53 and 54 were prepared by the method of Scheme VI.

### Example 53

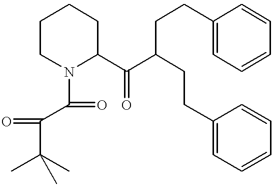

(2R, S)-2-({1-Oxo-[2-{2′-phenyl}ethyl]-4-phenyl}-butyl-1-(3 ,3-d imethyl-1 ,2-dioxobutyl)piperidine].

¹H NMR (CDCl₃, 300 MHz): δ1.40 (s, 9H); 1.36-2.29 (m, 9H); 2.62-2.82 (m, 4H); 3.39-3.52 (m, 2H); 5.18 (m, 1H); 5.31 (d, 1H, J=6.2); 5.42 (d, 1H, J=5.2); 7.42-7.26 (m, 1OH). Anal. Calcd. for C₂₉H₃₇NO₃: 0.5 H2O: C, 76.28; H, 8.39; N, 3.07. Found: C, 76.02; H, 8.29; N, 2.99. TLC: R_{f}=0.62 (20% EtOAc/hexane). Physical form: Clear oil

### Example 54

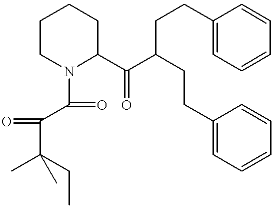

3,3-Dimethyl-1-[2-(2-phenethyl-4-phenylbutanoyl)piperidino]-1,2-pentanedione.

¹H NMR (CDCl₃, 400 MHz): δ0.94 (t, 3H, J=7.5); 1.24,1.28 (s, 3H each); 1.42-2.06 (m, 11H); 2.36 (d, 1H, J=13.0); 2.61-2.89 (dt, 1H, J=3.2, 12.9); 3.42 (brt, 1H, J=12.8); 5.08 (ddd, 1H, J=5.2, 7.2, 12.3); 5.32 (d, 1H, J=5.4); 7.16-7.32 (m, 1OH). Anal. Calcd. for C₃₀H₃₉NO₃: 0.7 H2O: C, 75.98; H, 8.59; N, 2.95. Found: C, 75.72; H, 8.28; N, 2.95. TLC: R_{f}=0.8 (20% EtOAc:Hexane). Physical form: Clear oil

### Example 55

Synthesis of pyrrolidine ketone compounds.

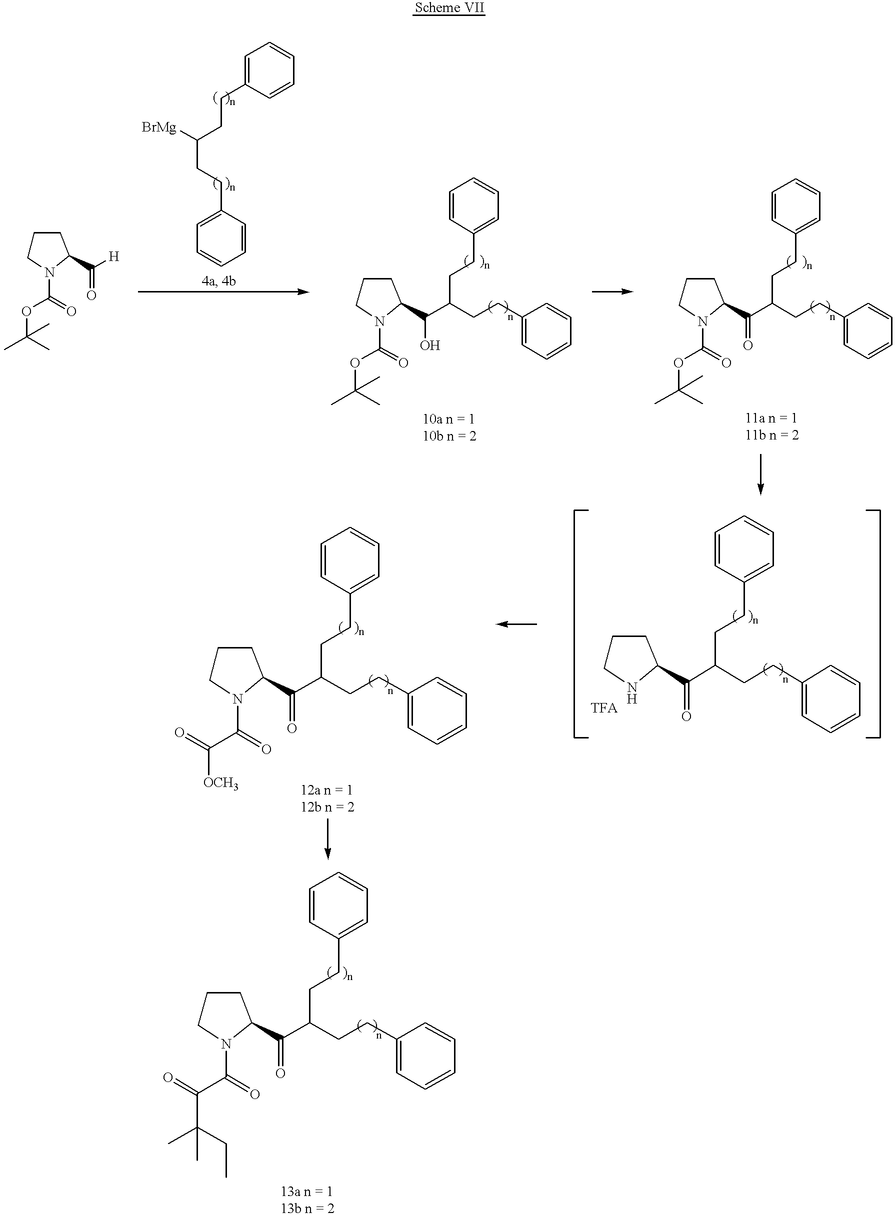

2-(1-Hydroxy-2-phenethyl-4-phenyl-butyl)-pyrroidine-1-carboxylic acid tert-butyl ester (10a).

To a solution of 0.5 g (2.5 mmol) of 2-formyl-pyrrolidine-l-carboxylic acid tert-butyl ester in anhydrous THF (20 mL) was added 5.OmL (2.5 mmol) of 4a, and the mixture was stirred at an ambient temperature for 16 hours. The solution was poured over a 1 N solution of hydrochloric acid and was extracted with EtOAc (2×100 mL). The organic phase was washed with saturated sodium bicarbonate, dried (MgSO₄) and evaporated to a clear oil which was subject to column chromatography (EtOAc/Hexanes, 1:3) to yield 0.18g (16.7%) of 1Oa as a clear oil. TLC R_{f}=0.6 (EtOAc/Hexanes, 1:3).

2-(2-Phenethyl-4-phenyl-butyryl)-pyrrolidine-1-carboxylic acid tert-butyl ester (11a).

To a solution of 0.22g (1.0 mmol) of pyridinium chlorochromate in anhydrous CH₂Cl₂ (15 mL) was added dropwise a solution of 0.2g (0.5 mmol) of 10a in anhydrous CH₂Cl₂ (5 mL), and the mixure was stirred at an ambient temperature for 16 hours. The solution was filtered and the resulting solution was evaporated to a yellow oil which was subject to column chromatography (EtOAc/Hexanes, 1:3) to yield 0.16g (76.2%) of Ila as a clear oil. TLC R_{f}=0.5 (EtOAc/Hexanes, 1:3).

Oxo-[2-(2-phenethyl-4-phenyl-butvrvl)-pyrrolidin-1-yl]-acetic acid methVl ester (12a).

To a solution of 0.18g (0.40 mmol) of 11a in CH₂Cl₂ (3 mL) was added dropwise 1 .OmL (8.77 mmol) of trifluoroacetic acid, and the mixture was allowed to stir for 2 hours. The solution was diluted with CH₂Cl₂ (1OmL) and cooled to 0° C. followed by dropwise addition of 0.7g (10.00 mmol) of triethylamine. After 5 minutes, to the solution was added dropwise 0.06g (0.5 mmol) of chlorooxoacetate, and the mixture was stirred for 2 hours. The solution was quenched with H₂O and extracted with CH₂Cl2 (2×2OOmL). The organic phase was dried (MgSO₄) and evaporated to a clear oil which was subject to column chromatography (EtOAc/Hexanes, 1:1) to yield 0.16g (98.2%) of 12a as a clear oil. TLC R_{f}=0.6 (EtOAc/Hexanes, 1:3).

3,3-Dimethyl-1-[2-(2-phenethyl-4-phenyl-butyryl)-pyrrolidin-1-yl]-pentane-1,2-dione (13a).

To a solution of 0.18g (0.45 mmol) of 12a in anhydrous THF (2 mL) was added dropwise at −78° C. a 1.0 M solution of 2.2 mL (2.20 mmol) of 1,1-dimethylpropyl magnesium chloride in THF, and the mixture was stirred for 3 hours at −78° C. The solution was poured over saturated ammonium chloride (50 mL) and extracted with EtOAc (3×100 mL). The organic phase was dried (MgSO₄) and evaporated to a clear oil which was subject to column chromatography (EtOAc/Hexanes, 1:3) to yield 0.14g (76.5%) of 13a as a clear oil. TLC R_{f}=0.5 (EtOAc/Hexanes, 1:3) ¹H NMR (CDCl₃, 400 MHz): δ0.82-0.90 (m, 3H); 1.12-1.33 (m, 6H); 1.59-1.79 (m, 7H); 2.00-2.20 (m, 3H); 2.40-2.70 (t, 5H); 3.41-3.52 (m, 2H); 4.63-4.64 (m,1H); 7.12-7.29 (m, 1OH); Anal. (C₂₉H₃₇NO₃) C, H, N.

3,3-Dimethyl-1-{2-[5-phenyl-2-(3-phenyl-propyl)-pentanol]-Dyrrolidin-1-yl}-pentane-1,2-dione (13b).

To a solution of 0.12g (0.28 mmol) of 12b in anhydrous THF (2 mL) was added dropwise at −78° C. a 1.0 M solution of 0.3 mL (0.3 mmol) of 1,1-dimethylpropyl magnesium chloride in THF, and the mixture was stirred for 3 hours at −78° C. The solution was poured over saturated ammonium chloride (50mL) and extracted with EtOAc (2×100 mL). The organic phase was dried (MgSO₄) and evaporated to a clear oil which was subject to column chromatography (EtOAc/Hexanes, 1:3) to yield 0.12g (91.6%) of 13b as a clear oil. TLC R_{f}=0.6 (EtOAc/Hexanes, 1:3) ¹H NMR (CDCl₃, 400 MHz): δ0.89 (t, 3H, J=7.5 Hz); 1.23 (s, 3H); 1.28 (s, 3H); 1.34-2.11 (m, 15H); 2.58-2.83 (m, 4H); 3.43 (dt, 1H, J=6.4,10.3 Hz); 3.56 (dt, 1H, J=7.1,10.3 Hz); 4.70 (dd, 1H, J=4.3 Hz); 7.12-7.31 (m, 1OH); Anal. (C₃₁H₄₁NO₃) C, H, N.

Examples 56 and 57 were prepared by the method of Scheme VII.

### Example 56

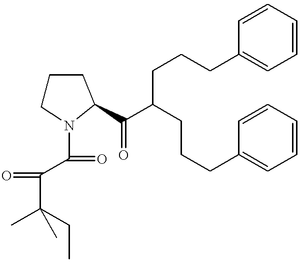

3,3-Dimethyl-1-{(2S)-2-[5-phenyl-2-(3-phenylpropyl)pentanoyl]-1-pyrrolidinyl}-1,2-pentanedione.

¹H NMR (CDCl₃, 400 MHz): δ0.89 (t, 3H, J=7.5); 1.23 (s, 3H); 1.28 (s, 3H); 1.34-2.11 (m, 15H); 2.58-2.83 (m, 4H); 3.43 (dt, 1H, J=6.4, 10.3); 3.56 (dt, 1H, J=7.1, 10.3); 4.70 (dd,1H, J=4.3); 7.12-7.31 (m, 1OH). Anal. Calcd. for C₃₁H₄₁NO₃: C, 78.28; H, 8.69; N, 2.94. Found: C, 78.10; H, 8.75; N, 2.90. TLC: R_{f}=0.52 (25% EtOAc/hexane). Physical form: Colorless oil

### Example 57

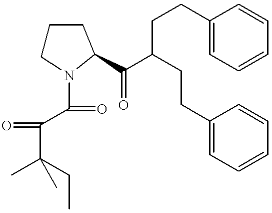

3,3-Dimethyl-1-{(2S)-2-[4-phenyl-2-(2-phenylethylbutanoyl]-1-pyrrolidinyl}-1,2-pentanedione.

¹H NMR (CDCl₃, 400 MHz): O 0.82-0.90 (m, 3H); 1.12-1.33 (m, 6H); 1.59-1.79 (m, 7H); 2.00-2.20 (m, 3H); 2.40-2.70 (t, 5H); 3.41-3.52 (m, 2H); 4.63-4.64 (m, 1H); 7.12-7.29 (m, 1OH). Anal. Calcd. for C₂₉H₃₇NO₃: 0.25 H₂O: C, 77.04; H, 8.36; N, 3.10. Found: C, 76.74; H, 8.25; N, 3.05.

### Example 58

Synthesis of amide compounds.

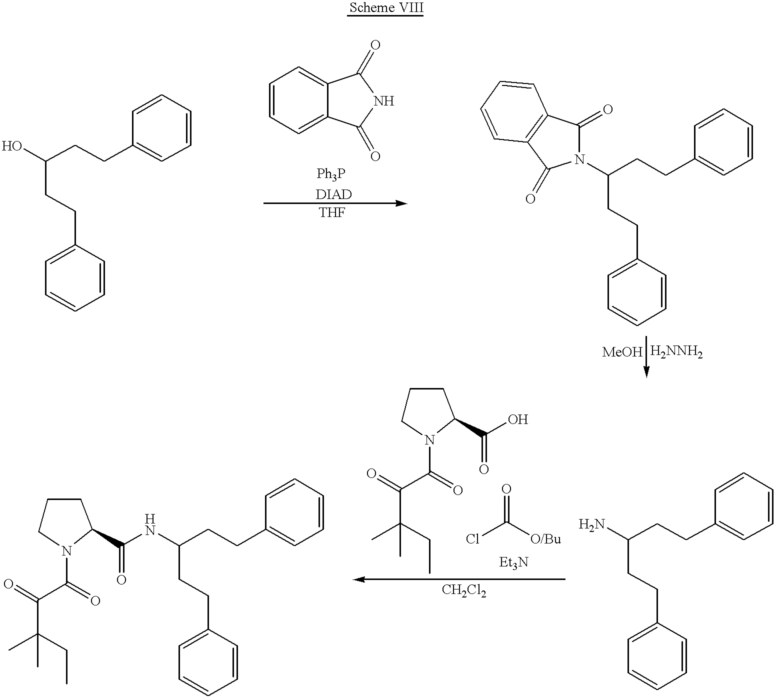

The general procedure for the synthesis of amide compounds is exemplified for, (2S)-[1-(3,3-Dimethyl-2-oxopentanoyl)pyrrolid i n-2-yl]-N-(1-phenylethyl-3-phenylpropyl)formamide, as follows:

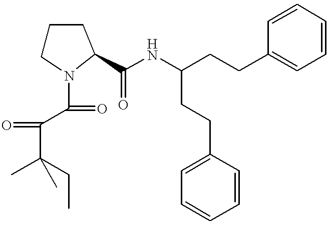

2-(1-Phenyl-3-phenyl-propyl)-isoindole-1,3-dione.

To a solution of 1,5-diphenyl-3-pentanol (0.65g, 2.7 mmol), phthalimide (0.40g, 2.7 mmol) and triphenylphosphine (0.75g, 2.8 mmol) in 17 mL THF was added dropwise DIAD (0.55g, 0.27 mmol) and the mixture stirred 1d. The mixture was then concentrated and the product purified on silica 15 gel using 9:1 hexane:ethyl acetate to a clear oil: 0.70g (70%); ¹H NMR (CDCl₃, 400 MHz): δ1.98-2.07(m, 2H); 2.47-2.63(m, 6H); 4.28-4.35(m, 1H); 7.03-7.26(m, 10H); 7.66-7.78(m, 4H). TLC: R_{f}=0.60 (EtOAc:Hexane 1:4).

1,5-Diphenyl-3-pentylamine.

To a solution of 1-Phenyl-3-phenyl-propyl)-isoindole-1,3-dione (0.68g, 1.8 mmol) in 20 mL methanol was added hydrazine monhydrate (0.92g, 18 mmol) and the mixture heated at reflux temperature for 3 h. The mixture was cooled to 4° C. and filtered. The filtrate was concentrated to yield product as a clear oil: 0.39g (89%); ¹H NMR (CDCl₃, 400 MHz): δ1.53-1.66(m, 2H); 1.71-1.84(m, 2H); 2.43-2.92(m, 7H); 7.12-7.32(m, 10H).

(2S)-[1-(3,3-Dimethyl-2-oxopentanoyl)pyrrolidin-2-yl]-N-(1-phenylethyl-3-phenylpropyl)formamide.

To a solution of 1-(3,3-Dimethyl-2-oxo-pentanoyl)-pyrrolidine-2-carboxylic acid (0.44g, 1.8 mmol) and triethylamine (0.19g, 1.8 mmol) in 7 mL dichloromethane under argon and cooled in an ice bath was added dropwise isobutyl chloroformate and the mixture stirred 5 min. At this time, a solution of 1,5-diphenyl-3-pentylamine (0.40g, 1.7 mmol) was added dropwise and the mixture stirred 1.5 h. allowing it to warm to room temperature. The mixture was then concentrated and the product purified on silica gel using 3:1 hexane ethyl acetate: 0.45g (58%); ¹H NfMR (CDCl₃, 400 MHz): δ0.86 (t, 3H, J=7.5); 1.23 (s, 6H); 1.72 (m, 4H); 1.82 (m, 2H); 1.95 (m, 2H); 2.10 (m, 1H); 2.42 (m, 1H); 2.63 (m, 4H); 3.47 (m, 2H); 4.02 (m, 1H); 4.56 (m, 1H); 6.58 (m,1H); 7.21 (m, 1OH). Anal. Calcd. for C₂₉H₃₈N₂O₃-H₂O: C, 72.47; H, 8.39; N, 5.83. Found: C, 72.09; H, 7.91; N, 5.71. TLC: R_{f}=0.70 (50% EtOAc/hexane). Physical form: Clear oil.

Examples 59 to 75 were prepared according to Scheme Vil.

### Example 59

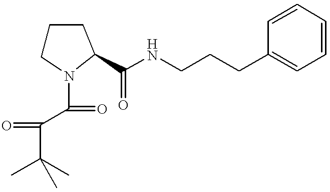

(2S)-[1-(3,3-Dimethyl-2-oxobutanoyl)pyrrolidin-2-yl]-N-(3-phenylpropyl)formamide.

¹H NMR (CDCl₃, 400 MHz): δ1.27 (s, 9H); 1.83 (m, 2H); 1.93 (m, 2H); 2.08 (m, 1H); 2.45 (m,1H); 2.66 (m, 2H); 3.28 (m, 2H); 3.42 (m, 2H); 4.54 (m,1H); 5.82 (m,1H); 7.26 (m, 5H). Anal. Calcd. for C₂₀H₂8N₂O₃: C, 69.74; H, 8.19; N, 8.13. Found: C, 68.74; H, 8.18; N, 7.91. TLC: R_{f}=0.40 (50% EtOAc/hexane). Physical form: Oil

### Example 60

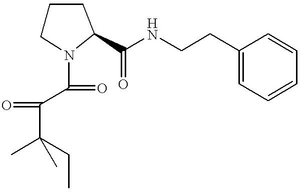

(2S)-[l -(3 ,3-Dimethyl-2-oxopentanoyl)pyrrolid in-2-yl]-N-(2-phenethyl)formamide.

¹H NMR (CDCl₃, 400 MHz): δ0.83 (t, 3H, J=7.5); 1.20 (s, 6H); 1.69 (m, 2H); 1.95 (m, 1H); 2.28 m,1H); 2.80 (m, 2H); 3.38 (m, 2H); 3.50 (m, 2H); 4.42 (m, 1H); 6.75 15 (br, 1H); 7.22-7.29 (m, 5H). Anal. Calcd. for C₂₀H₂₈N₂O₃: C, 69.74; H, 8.1 9; N, 8.13. Found: C, 69.49; H, 8.13; N, 8.13. TLC: R_{f}=0.50 (33% EtOAc/hexane). Physical form: Oil.

### Example 61

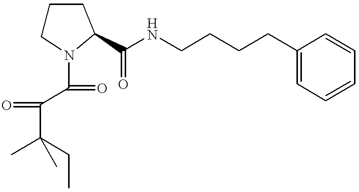

(2S)-[1-(3,3-Dimethyl-2-oxopentanoyl )pyrrol id in-2-yl]-N-(4-phenylbutyl)formamide.

¹H NMR (CDCl₃, 400 MHz): δ0.86 (t, 3H, J=7.5); 1.21 (s, 6H); 1.53-1.71 (m, 6H); 1.90 (m, 2H); 2.05 (m,1H); 2.41 (m,1H); 2.60 (m, 2H); 3.26 (m, 2H); 3.43 (m, 2H); 4.54 (m, 1H); 6.85 (br, 1H); 7.25-7.28 (m, 5H). Anal. Calcd. for C₂₂H₃₂N₂O₃: C, 70.94; H, 8.66; N, 7.52. Found: C, 70.79; H, 8.58; N, 7.42. TLC: R_{f}=0.50 (33% EtOAc/hexane). Physical form: Oil.

### Example 62

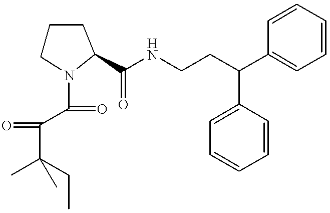

(2S)-[-(3,3-Dimethyl-2-oxopentanoyl)pyrrolidin-2-yl]-N-(3 ,3-diphenylpropyl)formamide.

¹H NMR (CDCl₃, 400 MHz): δ0.88 (t, 3H, J=7.5); 1.21 (s, 6H); 1.69 (m, 2H); 1. 88 (m, 2 H); 2.0 δ(m, 1H); 2.2 5 (m, 2 H); 2.3 0 (m, 1H); 3.2 0 (m, 2 H); 3.41 (m, 2 H); 3.9 7 (m, 1H); 4.50 (m, 1H); 7.20-7.28 (m, 1 OH). Anal. Calcd. for C₂₇H₃₄N₂O₃: C, 74.62; H, 7.89; N, 6.45. Found: C, 74.57; H, 7.85; N, 6.43. TLC: R_{f}=0.35 (25% EtOAc/hexane). Physical form: Oil.

### Example 63

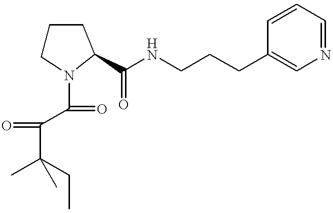

(2S)-[1-(3,3-Dimethyl-2-oxopentanoyl)pyrrolidin-2-yl]-N-(3-(3-pyridyl)propyl)formamide.

¹H NMR (CDCl₃, 400 MHz): δ0.86 (t, 3H, J=7.5); 1.22 (s, 6H); 1.71 (m, 2H); 1.83 (m, 2H); 1.94 (m, 2H); 2.02 (m, 1H); 2.35 (m, 1H); 2.62 (m, 2H); 3.28 (m, 2H); 3.46 (m, 2H); 4.56 (m, 1H); 7.10 (m, 1H); 7.50 (m, 1H); 8.44 (m, 2H). Anal. Calcd. for C₂₀H₂₉N₃O₃: 0.5 H₂O: C, 65.19; H, 8.21; N, 11.40. Found: C, 64.47; H, 8.01; N, 11.94. TLC: R_{f}=0.45 (25% EtOAc/hexane). Physical form: Oil.

### Example 64

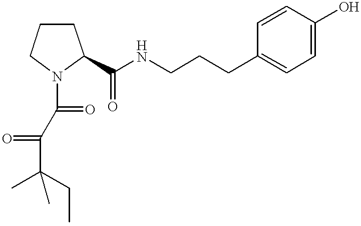

(2S)-[1-(3,3-Dimethyl-2-oxopentanoyl)pyrrolidin-2-yl]-N-[3-(4-hydroxyphenyl)propyl]formamide.

¹H NMR (CDCl₃, 400 MHz): δ0.88 (t, 3H, J=7.5); 1.24 (s, 6H); 1.70 (m, 6H); 1.78 (m, 2H); 2.05 (m, 1H); 2.41 (m, 1H); 2.54 (t, 2H); 3.24 (m, 2H); 3.44 (m, 2H); 4.53 (m,1H); 6.73 (d, 2H, J=8.30); 6.75 (br,1H); 6.98 (d, 2H, J=8.30). Anal. Calcd. for C₂lH₃₀N₂O₄: 0.5 H₂O: C, 65.77; H, 8.15; N, 7.30. Found: C, 65.63; H, 7.90; N, 7.05. TLC: R_{f}=0.45 (50% EtOAc/hexane). Physical form: Thick oil.

### Example 65

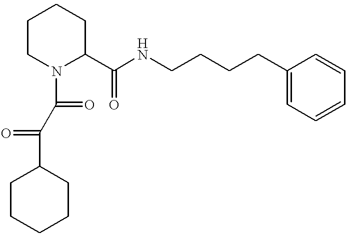

1-(2-Cyclohexyl-2-oxo-acetyl)-piperidine-2-carboxylic acid (4-phenyl-butyl)-amide.

¹H NMR (CDCl₃, 300 MHz): δ1.25-1.80 (m, 7H); 2.32-2.80 (m, 3H); 3.10-3.50 (m, 5H); 4.06 (m, 1H); 5.24 (m, 1H); 6.03 (m, 1H); 7.15-7.32 (m, 5H); 7.45-7.60 (m, 2H); 7.65-7.80 (m, 1H); 8.00-8.10 (m, 2H). Anal.Calcd. for C₂₄H₂₇N₂O₃: 0.5 H₂0; C, 71.98; H, 7.05; N, 6.99. Found: C, 71.95; H, 7.06; N, 7.12. TLC: R_{f}=0.20 (2:1 hexane:EtOAc) Physical form: Clear oil

### Example 66

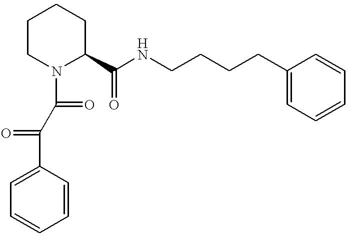

(2S)-1 [-(2-Oxo-2-phenylacetyl)(2-piperidyl )]-N-(4-phenylbutyl)-formamide.

¹H NMR (CDCl₃, 400 MHz): δ1.64-1.85 (m, 1OH); 2.05 (m, 1H); 2.38 (m, 3H); 3.31 (m, 2H); 3.45 (m, 1H); 4.05 (m, 1H); 5.22 (m, 1H); 6.08 (br, 1H); 6.55 (br, 1H); 7.25-7.97 (m, 1OH). Anal. Calcd. for C₂₄H₂₈N₂O₃: 0.7 H2O; C, 71.16; H, 7.31; N, 6.92. Found: C, 71.25; H, 7.14; N, 6.92. TLC: R_{f}=0.60 (1:1 Hexane/EtOAc). Physical form: Oil

### Example 67

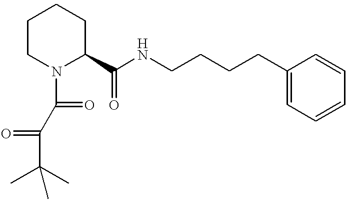

(2S)-[l -(3,3-Dimethyl-2-oxobutanoyl)(2-piperidyl)]-N-(4-phenylbutyl)formamide.

¹H NMR (CDCl₃, 400 MHz): 61.27 (s, 9H); 1.69-1.82 (m, 10H); 2.30-2.62 (m, 4H); 2.43 (m,1H); 2.50 (m, 2H); 3.80 (m,1H); 4.72 (m,1H); 5.95 (br,1H); 6.60 (br,1H); 7.20-7.56 (m, 5H). Anal. Calcd. for C₂₂H₃₂N₂O₃: C, 70.94; H, 8.66; N, 7.52. Found: C, 70.67, 8.63; N, 7.25. TLC: R_{f}=0.75 (1:1 Hexane/EtOAc). Physical form: Oil

### Example 68

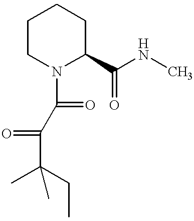

(2S)-[1-(3,3-Dimnethyl-2-oxopentanoyl)(2-piperidyl)]-N-rnethylformnamide. ¹H NMR (ODC1₃, 400 MHz): δ0.90(t, 3H, J=7.5); 1.22(s, 6H); 1.45(m, 2Hj); 1.72(m, 4H); 2.47(m, 2H); 2.83(m, 3H); 3.25(m, 2H); 5.08(m, 1H). Anal. Calcd. for: C, 61.10; H, 9.07; N, 10.18. Found: O, 61.12; H, 8.84; N, 10.01. TLC: R_{f} 0.40; 1:1 hexane:EtOAc. Physical form: Clear oil.

### Example 69

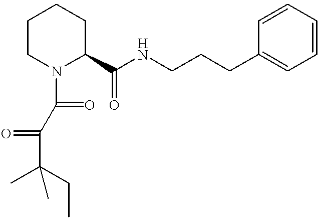

(2S)-[1-(3,3-Dimethyl-2-oxopentanoyl)(2-piperidyl)]-N-(3-phenylpropyl)formamide.

¹H NMR (CDCl₃, 400 MHz): δ0.90(t, 3H, J=7.5); 1.22(s, 6H); 1.45(m, 2H); 1.72(m, 4H); 1.83(m, 2H); 2.45(m, 2H); 2.65(m, 2H); 3.20(m, 2H); 3.30(m, 2H); 5.08(m, 1H); 6.02(bs,1H); 7.23(m, 5H). Anal. Calcd. for: C, 70.26; H, 8.68; N, 7.45. Found: C, 70.11; H, 8.67; N, 7.46. TLC: R_{f} =0.73; 1:1 hexane:EtOAc. Physical form: White solid.

### Example 70

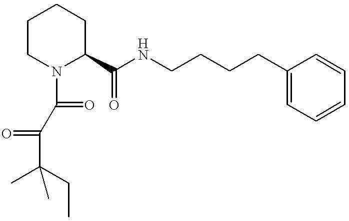

(2S)-[1-(3,3-Dimethyl-2-oxopentanoyl)(2-piperidyl)]-N-(4-phenylbutyl)formamide.

¹H NMR (CDCl₃, 400 MHz): δ0.90(t, 3H, J=7.5); 1.22(s, 6H); 1.54(m, 4H); 1.71(m, 6H); 2.45(m, 2H); 2.63(m, 2H); 3.20(m, 2H); 3.30(m, 2H); 5.04(m, 1H); 6.00(bs,1H); 7.23(m, 5H) Anal. Calcd. for: C, 70.39; H, 8.90; N, 7.14. Found: C, 70.38; H, 8.78; N, 7.11. TLC: R_{f} 0.77; 1:1 hexane:EtOAc. Physical form: Clear oil.

### Example 71

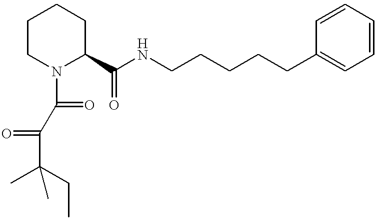

(2S)-[1-(3,3-Dimethyl-2-oxopentanoyl)(2-piperidyl)]-N-(5-phenylpentyl)formamide.

¹H NMR (CDCl₃, 400 MHz): δ0.90(t, 3H, J=7.5); 1.23(s, 6H); 1.40(m, 2H); 1.52(m, 4H); 1.71(m, 6H); 2.45(m, 2H); 2.61(m, 2H); 3.15(m, 2H); 3.28(m, 2H); 5.05(d, 1H, J=5.4); 5.96(bs, 1H); 7.21 (m, 5H). Anal. Calcd. for: C, 71.96; H, 9.06; N, 6.99. Found: C, 71.94; H, 9.10; N, 6.94. TLC: R_{f} 0.47; 2:1 hexane:EtOAc. Physical form: Clear oil.

### Example 72

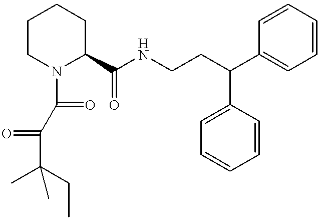

(2S)-[1-(3,3-Dimethyl-2-oxopentanoyl)piperidin-2-yl]-N-(3,3-diphenylpropyl)formamide.

¹H NMR (CDCl₃, 400 MHz): δ0.91(t, 3H, J=7.5); 1.23(s, 6H); 1.72(m, 6H); 2.28(m, 3H); 3.20(m, 3H); 4.00(m, 3H); 5.02(m, 1H); 5.98(bs, 1H); 7.24(m, 1OH) Anal. Calcd. for: C, 73.83; H, 8.15; N, 5.90. Found: C, 73.83; H, 8.10; N, 5.77. TLC: R_{f} 0.62; 2:1 hexane:EtOAc. Physical form: Clear oil.

### Example 73

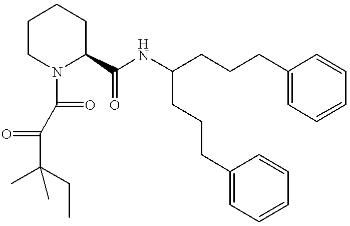

(2S)-[1-(3,3-Dimethyl-2-oxopentanoyl)piperidin-2-yl]-N-(1,7-diphenyl-4-heptyl)formamide.

¹H NMR (CDCl₃, 400 MHz): δ0.90(t, 3H, J=7.5); 1.23(m, 6H); 1.60(m, 14H); 2.40(m, 1H); 2.61 (m, 3H); 3.17(m, 1H); 4.00(m, 2H); 5.05(m,1H); 5.68(m, 1H); 7.25(m, 1OH). Anal. Calcd. for: C, 76.15; H, 8.79; N, 5.55. Found: C, 76.22; H, 8.82; N, 5.50. TLC: R_{f} 0.82; 2:1 hexane:EtOAc. Physical form: Clear oil.

### Example 74

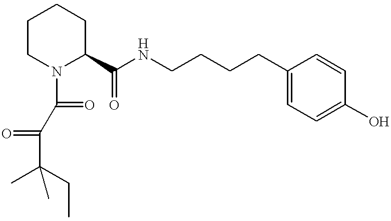

(2S)-[1-(3,3-Dimethyl-2-oxopentanoyl)(2-piperidyl)]-N-(4-{parahydroxyphenyl}butyl)formamide.

¹H NMR (CDCl₃, 400 MHz): δ0.90(t, 3H, J=7.5); 1.26(m, 8H); 1.50(m, 4H); 1.70(m, 4H); 2.55(m, 2H); 3.20(m, 3H); 4.13(m, 1H); 4.98(m, 2H); 5.05(m, 1H); 6.34(bs, 1H); 6.90(m, 4H). Anal. Calcd. for: C, 68.63; H, 8.51; N, 6.96. Found: C, 68.57; H, 8.51; N, 6.90. TLC: R_{f} 0.23; 2:1 hexane:EtOAc. Physical form: Clear oil.

### Example 75

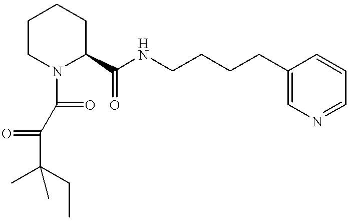

(2S)-[1-(3,3-Dimethyl-2-oxopentanoyl)(2-piperidyl)]-N-(4-{3-pyridyl}butyl)formamide.

¹H NMR (CDCl₃, 400 MHz): δ0.90(t, 3H, J=7.5); 1.22(m, 6H); 1.62(m, 12H); 2.45(m, 2H); 3.10(m, 1H); 3.32(m, 3H); 5.05(d, 1H, J=5.3); 6.05(bs, 1H); 7.21(m, 1H); 7.51(m, 1H); 8.43(m, 2H). Anal. Calcd. for: C, 67.40; H, 8.61; N, 10.72. Found: C, 67.49; H, 8.61; N, 10.68. TLC: R_{f} 0.18; 100% EtOAc. Physical form: Clear oil.

### Example 76

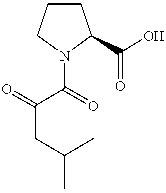

(2S)-1-(4-Methyl-2-oxopentanoyl )pyrrolid ine-2-carboxylic acid.

¹H NMR (CDCl₃, 400 MHz): δ0.88-0.97 (m, 6H); 1.82-2.18 (m, 5H); 2.70-2.83 (m, 2H); 3.78 (m, 2H); 4.90 (m, 1H); 7.98 (br,1H). Anal. Calcd. for Cl₁H₁₇NO₄: 0.25 H₂O: C, 57.01; H, 7.61; N, 6.01. Found: C, 57.30; H, 7.57; N, 5.91. Physical form: Semisolid.

### Example 77

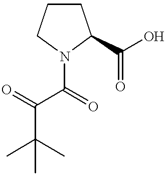

(2S)-I -(1 ,2-Dioxo-3,3-dimethylbutyl)-2-pyrrolidinecarboxylic acid.

¹H NMR (CDCl₃, 400 MHz): δ1.28 (s, 9H); 1.95-2.06 (m, 2H); 2.17-2.26 (m, 2H); 3.48-3.52 (m, 2H); 4.52 (d, 1H); 7.95 (br, 1H). Anal. Calcd. for CllHl₇NO₄: C, 58.14; H, 7.54; N, 6.16. Found: C, 58.40; H, 7.56; N, 6.14. Physical form: White solid.

### Example 78

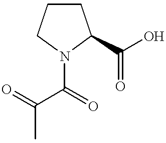

1-(2-Oxopropanoyl)pyrrolidine-2-carboxylic acid.

¹H NMR (CDCl₃, 400 MHz): δ1.98-2.10 (m, 1H); 2.21-2.30 (m, 1H); 2.41 (s, 3H); 3.66 (m, 2H); 3.77 (m, 2H); 4.87 (m, 1H); 10.46 (s, 1H). Anal. Calcd for C₈H,₁NO₄: 0.1 H₂O: C: 51.39; H: 6.04; N: 7.49. Found: C: 51.41; H: 6.27; N: 7.10. Physical form: Yellow gum.

### Example 79

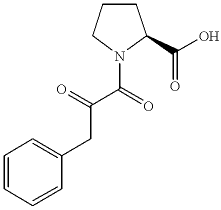

(2S)-1-(2-Oxo-3-phenylpropanoyl)pyrrolidine-2-carboxylic acid.

¹H NMR (CDCl₃, 400 MHz): δ1.86-2.26 (m, 4H); 3.59 (m, 2H); 4.06-4.16 (m, 2H); 4.50 (m, 1H); 7.18-7.33 (m, 5H); 8.12 (br, 1H). Anal. Calcd. for C₁₄H₁₅NO4: 0.25 H₂O; C, 63.27; H, 5.88; N, 5.27. Found: C, 63.33; H, 6.09; N, 4.49. Physical form: Yellow oil.

### Example 80

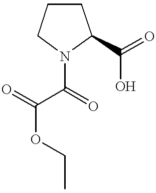

1-Ethoxyoxalyl-pyrrolidine-2-carboxylix acid.

¹H NMR (CDCl₃, 400 MHz): 61.31-1.40 (m, 3H); 1.87-2.49 (m, 4H); 3.61-3.87 (m, 2H); 4.23-4.36 (m, 2H); 4.58 and 4.93 (two sets of dd's of both rotamers, 1H); 9.62 (br.s, 1H). Anal. Calcd for C₉H₁₃N₁O₅: C, 50.23; H, 6.09; N, 6.51. Found: C, 50.1 1; H, 6.38; N, 6.04. Physical form: Yellow oil.

### Example 81

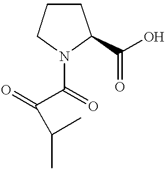

1 (2S-Methyl-Mtyccoey)2-oxobuetayl )pyrrolid ine-2-carboxylic acid.

¹H NMR (CDCl₃, 400 MHz): δ1.05-1.20 (m, 6H); 1.93-2.53 (m, 5 H); 3.53-3.76 (m,2H); 4.17-4.19 (m, 1H); 7.80 (br s, 1H). Anal Calcd for C₁₀H₁₅NO₄: 0.05 mol H₂O; C: 56.09; H:7.11; N: 6.54. Found: C: 55.91; H: 7.16; N: 6.36. Physical form: Oil

### Example 82

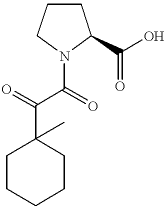

(2S)-1[2(1-Methylcyclohexyl)-2-oxoacetyl]pyrrolidine-2-carboxylic acid.

¹H NMR (CDCl₃, 400 MHz): δ1.29 (s, 3H); 1.30-1.40 (m, 6 H); 1.54-1.56 (m, 4H); 1.95-2.11 (m, 4H); 3.52-3.59 (m, 2H); 4.54 (dd, 1H, J=4,5); 10.30 (br s, 1H). Anal Calcd for C₁₄H₂,N₁O₄: C: 62.90; H: 7.92; N: 5.24. Found: C: 61.29; H: 7.75; N: 5.02. Physical form: Optically pure white solid.

### Example 83

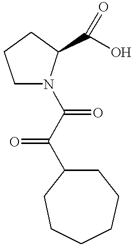

(2S)-1-(2-Cycloheptyl-2-oxoacetyl)pyrrolidine-2-carboxylic acid.

¹H NMR (CDCl₃, 400 MHz): δ1.27-1.41 (m, 10H); 1.51-1.98 (m, 4 H); 2.00-2.17 (m, 1H); 3.12-3.17 (m, 1H); 3.30-3.44 (m, 2H); 4.44 (dd, 1H, J=4,5). Anal. Calcd for C₁₄H₂₁NO₄: C: 62.90; H: 7.92; N: 5.24. Found: C: 62.74; H: 7.83; N: 5.11. Physical form: Optically pure white solid.

### Example 94

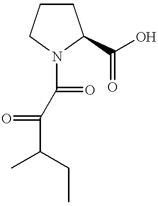

1-(3-Methyl-2-oxopentanoyl )pyrrol id ine-2-carboxyl ic acid.

NMR: ¹H NMR (CDCl3, 400 MHz): 0.88-0.96 (m, 3H); 1.06-1.14 (m, 3H); 1.25-1.50 (m, 1H); 1.67-2.1 1 (m, 3H); 2.19-2.24 (m, 2H); 3.20-3.30 (m, 1H); 3.60-3.78 (m, 2H); 4.59 (ti H, J=6.0); 9.47 (bs, 1H). TLC: R_{f}=0.36 (5% MeOH/EtOAc/5 drops HOAc). Anal: Calcd for: C, 58.14; H, 7.54; N, 6.16. Found: C, 58.32; H, 7.71; N, 6.04. Physical Form: Clear oil.

The invention being thus described, it will be obvious that the same may be varied in many ways. Such variations are not to be regarded as a departure from the spirit and scope of the invention and all such modification are intended to be included within the scope of the following claims.

We claim:

1. A method of treating a neurological activity in an animal, comprising:
administering to said animal an effective amount of a compound having an affinity for FKBP-type immunophilins according to formula II 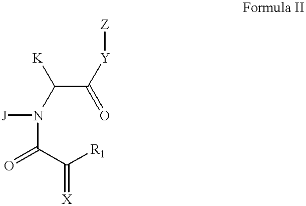
or a pharmaceutically acceptable salt thereof,
wherein Y is O, NH, or N-(C1-C4 alkyl);
wherein Z is hydrogen, CHL-Ar, (C1-C6)-straight or branched alkyl, (C2-C6)-straight or branched alkenyl, (C5-C7)-cycloalkyl, (C5-C7)-cycloalkenyl or Ar substituted (C1-C6)-alkyl or (C2-C6)-alkenyl, or 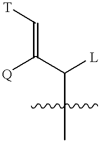
wherein L and Q are independently hydrogen, (C1-C6)-straight or branched alkyl or (C2-C6)-straight or branched alkenyl;
wherein T is Ar or substituted cyclohexyl with substituents at positions 3 and 4 which are independently selected from the group consisting of hydrogen, hydroxyl, O-(C1-C4)-alkyl or O-(C2-C4)-alkenyl and carbonyl;
wherein Ar is selected from the group consisting of 1-naphthyl, 2-naphthyl, 2-furyl, 3-furyl, 2-thienyl, 2-pyridyl, 3-pyridyl, 4-pyridyl and phenyl having one to three substituents which are independently selected from the group consisting of hydrogen, halo, hydroxyl, nitro, CF₃, (C1-C6)-straight or branched alkyl or (C2-C6)-straight or branched alkenyl, O-(C1-C4)-straight or branched alkyl or O-(C2-C4)-straight or branched alkenyl, O-benzyl, O-phenyl, amino and phenyl;
wherein R₁ is U; X is either oxygen or CH-U, provided that if R₁ is hydrogen, then X is CH-U, or if X is oxygen then R₁ is U;
wherein U is hydrogen, O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar;
wherein J is hydrogen or C1 or C2 alkyl or benzyl; K is (C1-C4)-straight or branched alkyl, benzyl or cyclohexylethyl; or wherein J and K may be taken together to form a 5-7 membered heterocyclic ring which may contain an oxygen (O), sulfur (S), SO or SO₂ substituted therein; and
wherein said neurological activity does not include amyotrophic lateral sclerosis.

2. The method of claim 1, wherein the neurological activity is selected from the group consisting of stimulation of damaged neurons, promotion of neuronal regeneration, prevention of neurodegeneration, and treatment of a neurological disorder.

3. The method of claim 2, wherein the neurological disorder is selected from the group consisting of peripheral neuropathies caused by physical injury or disease state, physical damage to the brain, physical damage to the spinal cord, stroke associated with brain damage, and neurological disorders relating to neurodegeneration.

4. The method of claim 3, wherein the neurological disorder is Alzheimer's Disease or Parkinson's Disease.

5. The method of claim 1, wherein J and K are taken together to form a 5 membered heterocyclic ring.

6. The method of claim 5,
wherein X is oxygen and R₁ is not hydrogen;
wherein U is O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar.

7. The method of claim 6, wherein
if R₁ is C1-C6 straight or branched alkyl, C2-C6 straight or branched alkenyl, C5-C7 cycloalkyl substituted with C1-C4 straight or branched alkyl or C2-C4 straight or branched alkenyl, or Ar, and Ar is 1-naphthyl, 2-naphthyl, 2-furyl, 3-furyl, 2-thienyl, 2-pyridyl, 3-pyridyl, 4-pyridyl, or phenyl;
then Z is not:
a C1-C6 alkyl or C2-C6 alkenyl substituted with substituted or unsubstituted 2-furyl, 3-furyl, 2-thienyl, 2-pyridyl, 3-pyridyl, 4-pyridyl, or phenyl; or
a C1-C6 alkyl or C2-C6 alkenyl substituted with C5-C7 cycloalkyl.

8. The method of claim 1, wherein J and K are taken together to form a 6 membered heterocyclic ring.

9. The method of claim 8,
wherein X is oxygen and R₁ is not hydrogen;
wherein U is O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar.

10. The method of claim 9, wherein
if Y is oxygen and RI is 1,1-dimethyl-1-propyl, then Z is not 3-cycloheylpropyl, 3-phenylpropyl, or 3-(3′,4,5-trimethoxyphenyl)propyl; and
if Y is oxygen and R₁ is 3′,4,5-trimethoxyphenyl, then Z is not 4-(4′-methoxyphenyl)butyl; and
if Y is oxygen and Z is ethyl, then R₁ is not methyl, ethyl, isopropyl, 2-methylpropyl, t-butyl, 1,1-dimethyl-1-propyl, phenyl, or benzyl.

11. The method according to claim 1, ,further comprising co-administering to said animal an effective amount of a neurotrophic factor selected from the group consisting of nerve growth factor, brain derived growth factor, glial derived growth factor, cilial neurotrophic factor, and neurotropin-3.

12. The method of claim 11, wherein the neurological activity is selected from the group consisting of stimulation of damaged neurons, promotion of neuronal regeneration, prevention of neurodegeneration and treatment of a neurological disorder.

13. The method of claim 12, wherein the neurological disorder is selected from the group consisting of peripheral neuropathies caused by physical injury or disease state, physical damage to the brain, physical damage to the spinal cord, and neurological disorders relating to neurodegeneration.

14. The method of claim 12, wherein the neurological disorder is Alzheimer's Disease or Parkinson's Disease.

15. The method of claim 11, wherein J and K are taken together to form a 5 membered heterocyclic ring.

16. The method of claim 11, wherein J and K are taken together to form a 6 membered heterocyclic ring.

17. A method for preventing neurodegeneration in an animal, comprising:
administering to said animal an effective amount of a compound having an affinity for FKBP-type immunophilins according to formula II 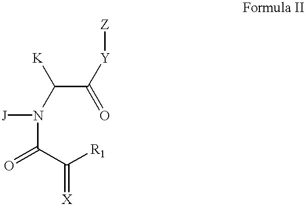
or a pharmaceutically acceptable salt thereof,
wherein Y is O, NH, or N-(C1-C4 alkyl);
wherein Z is hydrogen, CHL-Ar, (C1-C6)-straight or branched alkyl, (C2-C6)-straight or branched alkenyl, (C5-C7)-cycloalkyl, (C5-C7)-cycloalkenyl or Ar substituted (C1-C6)-alkyl or (C2-C6)-alkenyl, or 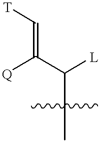
wherein L and Q are independently hydrogen, (C1-C6)-straight or branched alkyl or (C2-C6)-straight or branched alkenyl;
wherein T is Ar or substituted cyclohexyl with substituents at positions 3 and 4 which are independently selected from the group consisting of hydrogen, hydroxyl, O-(C1-C4)-alkyl or O-(C2-C4)-alkenyl and carbonyl;
wherein Ar is selected from the group consisting of 1-naphthyl, 2-naphthyl, 2-furyl, 3-furyl, 2-thienyl, 2-pyridyl, 3-pyridyl, 4-pyridyl and phenyl having one to three substituents which are independently selected from the group consisting of hydrogen, halo, hydroxyl, nitro, CF₃, (C1-C6)-straight or branched alkyl or (C2-C6)-straight or branched alkenyl, O-(C1-C4)-straight or branched alkyl or O-(C2-C4)-straight or branched alkenyl, O-benzyl, O-phenyl, amino and phenyl;
wherein R₁ is U; X is either oxygen or CH-U, provided that if R₁ is hydrogen, then X is CH-U, or if X is oxygen then R₁ is U;
wherein U is hydrogen, O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar;
wherein J is hydrogen or C1 or C2 alkyl or benzyl; K is (C1-C4)-straight or branched alkyl, benzyl or cyclohexylethyl; or wherein J and K may be taken together to form a 5-7 membered heterocyclic ring which may contain an oxygen (O), sulfur (S), SO or SO₂ substituted therein; and
wherein the neuronal degeneration is not caused by amyotrophic lateral sclerosis.

18. The method of claim 17, wherein J and K are taken together to form a 5 membered heterocyclic ring.

19. The method of claim 18,
wherein X is oxygen and R₁ is not hydrogen;
wherein U is O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar.

20. The method of claim 19, wherein
if R₁ is C1-C6 straight or branched alkyl, C2-C6 straight or branched alkenyl, C5-C7 cycloalkyl substituted with C1-C4 straight or branched alkyl or C2-C4 straight or branched alkenyl, or Ar, and Ar is 1-naphthyl, 2-naphthyl, 2-furyl, 3-furyl, 2-thienyl, 2-pyridyl, 3-pyridyl, 4-pyridyl, or phenyl;
then Z is not:
1) a C1-C6 alkyl or C2-C6 alkenyl substituted with substituted or unsubstituted 2-furyl, 3-furyl, 2-thienyl, 2-pyridyl, 3-pyridyl, 4-pyridyl, or phenyl; or
2) a C1-C6 alkyl or C2-C6 alkenyl substituted with C5-C7 cycloalkyl.

21. The method of claim 17, wherein J and K are taken together to form a 6 membered heterocyclic ring.

22. The method of claim 21,
wherein X is oxygen and R₁ is not hydrogen;
wherein U is O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar.

23. The method of claim 22, wherein
if Y is oxygen and R₁ is 1,1-dimethyl-1-propyl, then Z is not 3-cycloheylpropyl, 3-phenylpropyl, or 3-(3′,4,5-trimethoxyphenyl)propyl; and
if Y is oxygen and R₁ is 3′,4,5-trimethoxyphenyl, then Z is not 4-(4′-methoxyphenyl)butyl; and
if Y is oxygen and Z is ethyl, then R₁ is not methyl, ethyl, isopropyl, 2-methylpropyl, t-butyl, 1 ,1-dimthyl-1-propyl, phenyl, or benzyl.

24. The method of claim 17, further comprising co-administering an effective amount of a neurotrophic factor to prevent neurodegeneration selected from the group consisting of nerve growth factor, brain derived growth factor, glial derived growth factor, ciliary neurotrophic factor, and neurotropin-3.

25. A method for promoting neuronal regeneration and/or growth in an animal, comprising:
administering to said animal an effective amount of compound having an affinity for FKBP-type immunophilins according to formula II 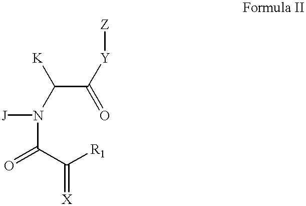
or a pharmaceutically acceptable salt thereof,
wherein Y is O, NH, or N-(C1-C4 alkyl);
wherein Z is hydrogen, CHL-Ar, (C1-C6)-straight or branched alkyl, (C2-C6)-straight or branched alkenyl, (C5-C7)-cycloalkyl, (G5-C7)-cycloalkenyl or Ar substituted (C1-C6)-alkyl or (C2-C6)-alkenyl, or 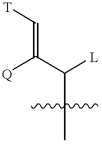
wherein L and Q are independently hydrogen, (C1-C6)-straight or branched alkyl or (C2-C6)-straight or branched alkenyl;
wherein T is Ar or substituted cyclohexyl with substituents at positions 3 and 4 which are independently selected from the group consisting of hydrogen, hydroxyl, O-(C1-C4)-alkyl or O-(C2-C4)-alkenyl and carbonyl;
wherein Ar is selected from the group consisting of 1-naphthyl, 2-naphthyl, 2-furyl, 3-furyl, 2-thienyl, 2-pyridyl, 3-pyridyl, 4-pyridyl and phenyl having one to three substituents which are independently selected from the group consisting of hydrogen, halo, hydroxyl, nitro, CF₃, (C1-C6)-straight or branched alkyl or (C2-C6)-straight or branched alkenyl, O-(C1-C4)-straight or branched alkyl or O-(C2-C4)-straight or branched alkenyl, O-benzyl, O-phenyl, amino and phenyl;
wherein R₁ is U; X is either oxygen or CH-U, provided that if R₁ is hydrogen, then X is CH-U, or if X is oxygen then R₁ is U;
wherein U is hydrogen, O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar; and
wherein J is hydrogen or Cl or C2 alkyl or benzyl; K is (C1-C4)-straight or branched alkyl, benzyl or cyclohexylethyl; or wherein J and K may be taken together to form a 5-7 membered heterocyclic ring which may contain an oxygen (O), sulfur (S), SO or SO2 substituted therein.

26. The method of claim 25, further comprising co-administering an effective amount of a neurotrophic factor to promote neuronal regeneration selected from the group consisting of nerve growth factor, brain derived growth factor, glial derived growth factor, and neurotropin-3.

27. The method of claim 25, wherein J and K are taken together to form a 5 membered heterocyclic ring.

28. The method of claim 27,
wherein X is oxygen and R₁ is not hydrogen;
wherein U is O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar.

29. The method of claim 28, wherein
if R₁ is C1-C6 straight or branched alkyl, C2-C6 straight or branched alkenyl, C5-C7 cycloalkyl substituted with C1-C4 straight or branched alkyl or C2-C4 straight or branched alkenyl, or Ar, and Ar is 1-naphthyl, 2-naphthyl, 2-furyl, 3-furyl, 2-thienyl, 2-pyridyl, 3-pyridyl, 4-pyridyl, or phenyl;
then Z is not:
1) a C1-C6 alkyl or C2-C6 alkenyl substituted with substituted or unsubstituted 2-furyl, 3-furyl, 2-thienyl, 2-pyridyl, 3-pyridyl, 4-pyridyl, or phenyl; or
2) a C1-C6 alkyl or C2-C6 alkenyl substituted with C5-C7 cycloalkyl.

30. The method of claim 25, wherein J and K are taken together to form a 6 membered heterocyclic ring.

31. The method of claim 30,
wherein X is oxygen and R₁ is not hydrogen;
wherein U is O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar.

32. The method of claim 31, wherein
if Y is oxygen and R₁ is 1,1-dimethyl-1-propyl, then Z is not 3-cycloheylpropyl, 3-phenylpropyl, or 3-(3′,4,5-trimethoxyphenyl)propyl; and
if Y is oxygen and R₁ is 3′,4,5-trimethoxyphenyl, then Z is not 4-(4′-methoxyphenyl)butyl; and
if Y is oxygen and Z is ethyl, then R₁ is not methyl, ethyl, isopropyl, 2-methylpropyl, t-butyl, 1,1-dimthyl-1-propyl, phenyl, or benzyl.

33. A method for stimulating the growth of at least one damaged peripheral nerve, comprising:
administering to said damaged peripheral nerve an effective amount of a compound having an affinity for FKBP-type immunophilins according to formula II 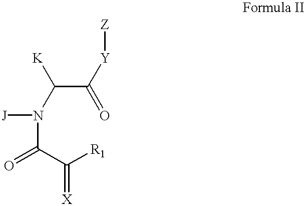
or a pharmaceutically acceptable salt thereof,
wherein Y is O, NH, or N-(C1-C4 alkyl);
wherein Z is hydrogen, CHL-Ar, (C1-C6)-straight or branched alkyl, (C2-C6)-straight or branched alkenyl, (C5-C7)-cycloalkyl, (C5-C7)-cycloalkenyl or Ar substituted (C2-C6)-alkyl or alkenyl, or 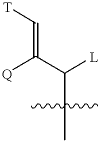
wherein L and Q are independently hydrogen, (C1-C6)-straight or branched alkyl or (C2-C6)-straight or branched alkenyl;
wherein T is Ar or substituted cyclohexyl with substituents at positions 3 and 4 which are independently selected from the group consisting of hydrogen, hydroxyl, O-(C1-C4)-alkyl or O-(C2-C4)-alkenyl and carbonyl;
wherein Ar is selected from the group consisting of 1-naphthyl, 2-naphthyl, 2-furyl, 3-furyl, 2-thienyl, 2-pyridyl, 3-pyridyl, 4-pyridyl and phenyl having one to three substituents which are independently selected from the group consisting of hydrogen, halo, hydroxyl, nitro, CF₃, (C1-C6)-straight or branched alkyl or (C2-C6)-straight or branched alkenyl, O-(C1-C4)-straight or branched alkyl or O-(C2-C4)-straight or branched alkenyl, O-benzyl, O-phenyl, amino and phenyl;
wherein R₁ is U; X is either oxygen or CH-U, provided that if R₁ is hydrogen, then X is CH-U, or if X is oxygen then R₁ is U;
wherein U is hydrogen, O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar; and
wherein J is hydrogen or C1 or C2 alkyl or benzyl; K is (C1-C4)-straight or branched alkyl, benzyl or cyclohexylethyl; or wherein J and K may be taken together to form a 5-7 membered heterocyclic ring which may contain an oxygen (O), sulfur (S), SO or SO2 substituted therein.

34. The method of claim 33, further comprising co-administering an effective amount of a neurotrophic factor to stimulate growth of the damaged peripheral nerve selected from the group consisting of nerve growth factor, brain derived growth factor, glial derived growth factor, and neurotropin-3.

35. The method of claim 33, wherein J and K are taken together to form a 5 membered heterocyclic ring.

36. The method of claim 35,
wherein X is oxygen and R, is not hydrogen;
wherein U is O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(Cl1-C4)-alkyl or (C2-C4)-alkenyl]-Ar or Ar.

37. The method of claim 36, wherein
if R₁ is C1-C6 straight or branched alkyl, C2-C6 straight or branched alkenyl, C5-C7 cycloalkyl substituted with C1-C4 straight or branched alkyl or C2-C4 straight or branched alkenyl, or Ar, and Ar is 1-naphthyl, 2-naphthyl, 2-furyl, 3-furyl, 2-thienyl, 2-pyridyl, 3-pyridyl, 4-pyridyl, or phenyl;
then Z is not:
1) a C1-C6 alkyl or C2-C6 alkenyl substituted with substituted or unsubstituted 2-furyl, 3-furyl, 2-thienyl, 2-pyridyl, 3-pyridyl, 4-pyridyl, or phenyl; or
2) a C1-C6 alkyl or C2-C6 alkenyl substituted with C5-C7 cycloalkyl.

38. The method of claim 33, wherein J and K are taken together to form a 6 membered heterocyclic ring.

39. The method of claim 38,
wherein X is oxygen and R₁ is not hydrogen;
wherein U is O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar.

40. The method of claim 39, wherein
if Y is oxygen and R₁ is 1 ,1-dimethyl-1-propyl, then Z is not 3-cycloheylpropyl, 3-phenylpropyl, or 3-(3′,4,5-trimethoxyphenyl)propyl; and
if Y is oxygen and R₁ is 3′,4,5-trimethoxyphenyl, then Z is not 4-(4′-methoxyphenyl)butyl; and
if Y is oxygen and Z is ethyl, then R₁ is not methyl, ethyl, isopropyl, 2-methylpropyl, t-butyl, 1 ,1-dimthyl-1-propyl, phenyl, or benzyl.

41. A method for stimulating neurite outgrowth by a nerve cell, comprising:
administering to said nerve cell an effective amount of compound having an affinity for FKBP-type immunophilins according to formula II 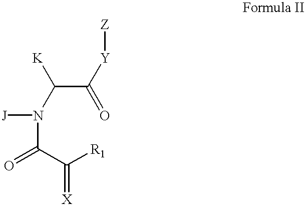
or a pharmaceutically acceptable salt thereof,
wherein Y is O, NH, or N-(C1-C4 alkyl);
wherein Z is hydrogen, CHL-Ar, (C1-C6)-straight or branched alkyl, (C2-C6)-straight or branched alkenyl, (C5-C7)-cycloalkyl, (C5-C7)-cycloalkenyl or Ar substituted (C1-C6)-alkyl or (C2-C6)-alkenyl, or 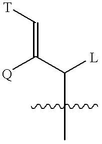
wherein L and Q are independently hydrogen, (C1-C6)-straight or branched alkyl or (C2-C6)-straight or branched alkenyl;
wherein T is Ar or substituted cyclohexyl with substituents at positions 3 and 4 which are independently selected from the group consisting of hydrogen, hydroxyl, O-(C1-C4)-alkyl or O-(C2-C4)-alkenyl and carbonyl;
wherein Ar is selected from the group consisting of 1-naphthyl, 2-naphthyl, 2-furyl, 3-furyl, 2-thienyl, 2-pyridyl, 3-pyridyl, 4-pyridyl and phenyl having one to three substituents which are independently selected from the group consisting of hydrogen, halo, hydroxyl, nitro, CF₃, (C1-C6)-straight or branched alkyl or (C2-C6)-straight or branched alkenyl, O-(C1-C4)-straight or branched alkyl or O-(C2-C4)-straight or branched alkenyl, O-benzyl, O-phenyl, amino and phenyl;
wherein R₁ is U; X is either oxygen or CH-U, provided that if R₁ is hydrogen, then X is CH-U, or if X is oxygen then R₁ is U;
wherein U is hydrogen, O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar; and
wherein J is hydrogen or C1 or C2 alkyl or benzyl; K is (C1-C4)-straight or branched alkyl, benzyl or cyclohexylethyl; or wherein J and K may be taken together to form a 5-7 membered heterocyclic ring which may contain an oxygen (O), sulfur (S), SO or SO2 substituted therein.

42. The method of claim 41, further comprising co-administering an effective amount of a neurotrophic factor to stimulate neurite outgrowth selected from the group consisting of nerve growth factor, brain derived growth factor, glial derived growth factor, and neurotropin-3.

43. The method of claim 41, wherein J and K are taken together to form a 5 membered heterocyclic ring.

44. The method of claim 43,
wherein X is oxygen and R₁ is not hydrogen;
wherein U is O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(Cl -C4)-alkyl or (C2-C4)-alkenyl]-Ar or Ar.

45. The method of claim 44, wherein
if R₁ is C1-C6 straight or branched alkyl, C2-C6 straight or branched alkenyl, C5-C7 cycloalkyl substituted with C1-C4 straight or branched alkyl or C2-C4 straight or branched alkenyl, or Ar, and Ar is 1-naphthyl, 2-naphthyl, 2-furyl, 3-furyl, 2-thienyl, 2-pyridyl, 3-pyridyl, 4-pyridyl, or phenyl;
then Z is not:
1) a C1-C6 alkyl or C2-C6 alkenyl substituted with substituted or unsubstituted 2-furyl, 3-furyl, 2-thienyl, 2-pyridyl, 3-pyridyl, 4-pyridyl, or phenyl; or 2) a C1-C6 alkyl or C2-C6 alkenyl substituted with C5-C7 cycloalkyl.

46. The method of claim 41, wherein J and K are taken together to form a 6 membered heterocyclic ring.

47. The method of claim 46,
wherein X is oxygen and R₁ is not hydrogen;
wherein U is O-(C1-C4)-straight or branched alkyl or O-(C2-C4)-straight or branched alkenyl, C1-C6-straight or branched alkyl, or C2-C6-straight or branched alkenyl, C5-C7-cycloalkyl or (C5-C7)-cycloalkenyl substituted with (C1-C4)-straight or branched alkyl or (C2-C4)-straight or branched alkenyl, 2-indolyl, 3-indolyl, [(C1-C4)-alkyl or (C2-C4)-alkenyl]-Ar or Ar.

48. The method of claim 47, wherein
if Y is oxygen and R₁ is 1,1-dimethyl-1-propyl, then Z is not 3-cycloheylpropyl, 3-phenylpropyl, or 3-(3′,4,5-trimethoxyphenyl)propyl; and
if Y is oxygen and R₁ is 3′,4,5-trimethoxyphenyl, then Z is not 4-(4′-methoxyphenyl)butyl; and
if Y is oxygen and Z is ethyl, then R₁ is not methyl, ethyl, isopropyl, 2-methylpropyl, t-butyl, 1 ,1-dimthyl-1-propyl, phenyl, or benzyl.

49. The method of claim 41, wherein said method is used to treat a patient who is suffering from or has suffered from Alzheimer's disease, Parkinson's disease, stroke, ischemia associated with stroke, neural paropathy, other neural degenerative diseases, motor neuron diseases, sciatic nerve crush, spinal cord injuries, or facial nerve crush.
